(19) United States
(12) Patent Application Publication
(10) Pub. No.: US 2024/0050334 A1
(43) Pub. Date: Feb. 15, 2024

(54) Oral Care Compositions and Methods

(71) Applicant: Colgate-Palmolive Company, New York, NY (US)

(72) Inventors: Lyndsay SCHAEFFER-KORBYLO, Flemington, NJ (US); Carl MYERS, Wayne, NJ (US); Gokul GOVINDARAJU, Somerville, NJ (US); Reeba PAUL, Edison, NJ (US)

(73) Assignee: Colgate-Palmolive Company, New York, NY (US)

(21) Appl. No.: 18/258,267

(22) PCT Filed: Dec. 20, 2021
(86) PCT No.: PCT/US2021/064402

§ 371 Date: Jun. 19, 2023

Related U.S. Application Data
(60) Provisional application No. 63/128,678, filed on Dec. 21, 2020.

(51) Int. Cl. A61K 8/24; A61K 8/21; A61K 8/19; A61K 8/44; A61K 33/16; A61K 33/42; A61K 47/02; A61K 47/18; A61Q 11/00

(52) U.S. Cl. CPC A61K 8/24; A61K 8/21; A61K 8/19; A61K 8/44; A61K 33/16; A61K 33/42; A61K 47/02; A61K 47/183; A61Q 11/00

(57) ABSTRACT

The application relates to novel aqueous oral care compositions useful for combining and delivering incompatible stannous fluoride or stannous chloride or stannous pyrophosphate, potassium salts, and an amino acid (e.g., a basic amino acid) in a high-water composition, for example, to provide effective caries prevention, protection against dental erosion, and relief from dental hypersensitivity. Additionally, oral care compositions described here useful in order to naturally promote nitrate reduction from the oral microbiome, which can eventually result in systemic increases of nitric oxide in blood plasma, and can form part of an overall regimen to maintain or control blood pressure. Compositions comprise stannous fluoride, stannous chloride or stannous pyrophosphate, nitric acid or a water-soluble nitrate salt such as potassium nitrate, a water-soluble alkali metal polyphosphate such as tetrasodium pyrophosphate or trisodium polyphosphate, a basic amino aid such as arginine and more than 10 wt % water.

### FIELD OF THE INVENTION

This application relates to novel aqueous oral care compositions useful for combining and delivering incompatible stannous fluoride or stannous chloride or stannous pyrophosphate, potassium salts, and an amino acid (e.g., a basic amino acid) in a high-water composition, for example, to provide effective caries prevention, protection against dental erosion, and relief from dental hypersensitivity. Additionally, oral care compositions described here useful in order to naturally promote nitrate reduction from the oral microbiome, which can eventually result in systemic increases of nitric oxide in blood plasma, and can form part of an overall regimen to maintain or control blood pressure.

### BACKGROUND

Dental plaque is a sticky biofilm or mass of bacteria that is commonly found between the teeth, along the gum line, and below the gum line margins. Dental plaque can give rise to dental caries and periodontal problems such as gingivitis and periodontitis. Dental caries tooth decay or tooth demineralization caused by acid produced from the bacterial degradation of fermentable sugar. Consequently, the presence of biofilm can be detrimental to the overall health one's oral cavity. And while oral care is often thought of simply in terms of maintaining oral health and preventing cavities, gingivitis or malodor, the oral cavity also plays a role in the overall health of the body.

One way to enhance or improve systemic health, e.g., by improving the health of the oral cavity, is to increase the amount circulating nitric oxide in plasma. In turn, “enterosalivary nitrate cycling” refers to the mechanism whereby dietary nitrate is reduced to nitrite by salivary bacteria. Without being bound by theory, nitrite which is ingested can then be converted to nitric oxide by bacteria in the gut and this nitric oxide can then diffuse into the circulatory system. Plasma nitric oxide can serve as a vasodilator and lead to reductions in blood pressure. Harnessing this potential and promoting the growth and metabolism of salivary nitrate reducing bacteria can lead to meaningful reductions in blood pressure. Consequently, compounds that can potentially decrease biofilm, and potentially increase the amount circulating nitric oxide in individual's system, could be beneficial in terms of improving both oral and systemic health, e.g., by maintaining or controlling blood pressure.

Stannous ion sources, such as stannous fluoride and stannous chloride, are known for use in clinical dentistry with a history of therapeutic benefits over forty years, and can have use in reducing certain bacterial growth in the oral cavity. However, until recently, the popularity of stannous ion sources has been limited by the instability in aqueous solutions. The instability of stannous salts in water is primarily due to the reactivity of the stannous ion (Sn²⁺). Stannous salts readily hydrolyze at a pH above 4, resulting in precipitation from solution. It has traditionally been thought that this formation of insoluble stannous salts results in a loss of therapeutic properties.

One common way to overcome the stability problems associated with stannous ions is to limit the amount of water in the composition to very low levels, or to use a dual phase system. Both of these solutions to the stannous ion problem have drawbacks. Low water oral care compositions can be difficult to formulate with desired rheological properties, and dual-phase compositions are considerably more expensive to manufacture and package. Thus, it is preferable to formulate a high-water composition which uses an alternative means to maintain stable efficacious stannous ion concentrations.

However, while it may be beneficial, e.g., for purposes of encouraging or enhancing enterosalivary nitrate cycling in the oral cavity, to prepare formulations with potassium and stannous salts, it has also been reported that aqueous oral care compositions comprising unstabilized stannous ion and nitrate ion together may form potentially toxic species such as nitrite ion and nitrosamines, due to the reduction of the nitrate ion by the stannous ion. To avoid this issue, two-component composition have been suggested with the stannous ion source and the nitrate ion source in separate components. One way this can be potentially resolved, in a single-phase aqueous composition, is by strictly controlling the molar ratio of solvated nitrate ion to solvated stannous ion of less than 2:1 at a pH of 3 to 6. Another way this can be potentially resolved, again in a single-phase composition, is by stabilizing the stannous ion with a chelant, such as citric acid or polyphosphates such as tripolyphosphate, in moderate water compositions (e.g., 20-65% water),

However, one potentially drawback may be the further difficulty of having fluoride ions in an oral care composition tending to precipitate out of solution when potassium nitrate is present, due to the low solubility of ionic fluoride sources. Some approaches to this problem incorporate the use of monofluorophosphate salts rather than fluoride salts as fluoride ion sources.

Many references do not take issue with or seem to be aware of the unique formulation difficulties which may be encountered in the preparation of formulations comprising stannous salts, fluoride salts, and polyphosphate. Other reference disclosing similar compositions avoid the issues by resorting to dual-component manufactures.

There is thus a need for novel oral compositions and methods that provide stable formulations of stannous fluoride or stannous chloride and potassium salts, which, in turn, can also be benefit systemic health, e.g., by helping to maintain or control blood pressure.

### BRIEF SUMMARY

In one aspect, the oral care compositions described herein contemplate compositions that comprise stannous fluoride or stannous chloride or stannous pyrophosphate, nitric acid or a soluble nitrate salt (e.g., KNO₃), a basic amino acid (e.g., arginine) and an alkali metal polyphosphate salt in high-water oral care composition. In one aspect, the compositions function as a system for the promotion of enterosalivary nitrate metabolism which can help to reduce, maintain, and/or control blood pressure, e.g., by increasing the levels of nitric oxide in a subject's circulating blood plasma.

Without being bound by theory, a number of oral bacterial species have been identified as being involved in enterosalivary nitrate metabolism, and the compositions described herein (e.g., Composition 1.0 et seq) are believed to be able to increase the presence of one or more of oral bacterial species involved in enterosalivary nitrate metabolism. In one aspect, the compositions described herein (e.g., Compositions 1.0 et seq) can increase the presence of one or more of the following bacterial species believed to be involved in enterosalivary nitrate metabolism: Actinomyces naeslundii, Actinomyces odontolyticus, Actinomyces oris, Actinomyces viscosus, Bacillus brevis, Capnocytophaga sputigena, Corynebacterium durum, Corynebacterium matruchotii, Eikenella corrodens, Granulicatella adiacens, Haemophilus parainfluenzae, Haemophilus segnis, Microbacterium oxydans, Neisseria flavescens, Neisseria sicca, Neisseria subflava, Prevotella melaninogenica, Prevotella salivae, Priopionibacterium acnes, Rothia denticariosa, Rothia mucilaginosa, Staphylococcus epidermidis, Staphylococcus hemolyticus, Selenomonas noxia, Veillonella dispar, Veillonella parvula, and Veillonella atypica. Without being bound by theory, it is believed that by increasing the presence of one or more of the oral bacterial species involved in enterosalivary nitrate metabolism, this can eventually contribute to the increase of a subject's plasma nitric oxide levels.

Again, without being bound by theory, the compositions described herein are believed to be able to deliver substrates to oral bacteria, where the substrates are designed to target and promote oral bacteria capable of metabolizing nitrate. In turn, the administration of the compositions described herein (e.g., any of Composition 1.0 et seq) can shift the balance of the oral bacterial community to one where more nitrate reduction occurs, which will lead to increased nitrite being ingested and passed into the gut, and then further reduced to nitric oxide.

It is believed that the community composition of the oral cavity is considerably more stable than other sites of the body and, therefore, repeated, prolonged exposure is required in order to create meaningful bacterial community shifts. The use of oral care formulations described herein allows for delivery of ingredients designed to feed the nitrate reducing bacteria in the oral cavity which allows for repeated application over extended periods of time, and promoting shifts in the oral bacterial community.

Without being bound by theory, the compositions described herein (e.g., any of Composition 1.0 et seq) are believed to provide active ingredients that can naturally promote nitrate reduction from the oral microbiome. For example, stannous salts, such as stannous fluoride, are known antimicrobial agents in the oral care field and has been demonstrated to slow or halt bacterial metabolism. In this system, the reduction in bacterial metabolism is believed to aid in the ability of other ingredients to influence bacterial activity. Furthermore, potassium salts, such as KNO₃, are believed to provide a short-term source of nitrate to help promote overall nitrate metabolism within the oral bacterial community. A basic amino acid, such arginine, serves as a starting substrate in the nitrite reduction pathway that ultimately leads to the production of nitric oxide, the desired endpoint of enterosalivary nitrate cycling. By providing exogenous arginine, for example, and without being bound by theory, the oral care compositions described herein are believed to promote the long term nitrate reducing capacity of an individual. This, in turn, is believed to lead to increased nitrate cycling and, ultimately, improved blood pressure control via increasing the levels of circulating nitric oxide in the blood plasma.

U.S. application Ser. No. 16/840,857, incorporated by reference herein in its entirety, discloses the surprising discovery that a combination of stannous fluoride or stannous chloride, nitric acid or a soluble nitrate salt, and an alkali metal polyphosphate salt in high-water oral care composition results in stability of stannous, fluoride and nitrate in solution.

The disclosure further provides single-component oral care composition packages comprising the compositions disclosed herein.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight relative to the total composition. The amounts given are based on the active weight of the material.

As is usual in the art, the compositions described herein are sometimes described in terms of their ingredients, notwithstanding that the ingredients may disassociate, associate or react in the formulation. Ions, for example, are commonly provided to a formulation in the form of a salt, which may dissolve and disassociate in aqueous solution. It is understood that the invention encompasses both the mixture of described ingredients and the product thus obtained.

In a first aspect, the present disclosure provides a single-component oral care composition (Composition 1.0) comprising:
(i) stannous fluoride and/or stannous chloride and/or stannous pyrophosphate;
(ii) nitric acid or a water-soluble nitrate salt (e.g., potassium nitrate);
(iii) a water-soluble alkali metal polyphosphate (e.g., sodium or potassium pyrophosphate or tripolyphosphate);
(iv) an amino acid (e.g., a basic amino acid) (e.g., arginine) (e.g., in free or salt form); and
(v) more than 10% water, by weight of the composition.

For example, the disclosure provides embodiments of Composition 1.0 as follows:
1.1 Composition 1.0, wherein the water-soluble nitrate salt is selected from an alkali or alkaline earth metal nitrate, or zinc nitrate, silver nitrate, or ammonium nitrate.
1.2 Composition 1.1, wherein the water-soluble nitrate salt is an alkali metal nitrate salt or an alkaline earth metal nitrate salt.
1.3 Composition 1.2, wherein the nitrate salt is selected from lithium nitrate, sodium nitrate, potassium nitrate, magnesium nitrate, and calcium nitrate.
1.4 Composition 1.3, wherein the nitrate salt is potassium nitrate.
1.5 Any foregoing composition, wherein the water-soluble alkali metal polyphosphate is selected from a pyrophosphate, tripolyphosphate, tetraphosphate or hexametaphosphate.
1.6 Any foregoing composition, wherein the water-soluble alkali metal polyphosphate is a sodium or potassium polyphosphate.
1.7 Any foregoing composition, wherein the water-soluble alkali metal polyphosphate is selected from sodium pyrophosphate, potassium pyrophosphate, sodium tripolyphosphate and potassium tripolyphosphate.
1.8 Composition 1.7, wherein the sodium pyrophosphate salt is selected from sodium acid pyrophosphate (i.e., disodium pyrophosphate) and tetrasodium pyrophosphate.
1.9 Any foregoing composition, wherein the water-soluble nitrate salt is potassium nitrate and the water-soluble alkali metal polyphosphate salt is tetrasodium pyrophosphate.
1.10 Any foregoing composition, wherein the composition comprises a molar ratio of alkali metal polyphosphate (e.g., tetrasodium pyrophosphate) to stannous fluoride or stannous chloride or stannous pyrophosphate of at least 1:1, e.g., 1:1 to 5:1, or 1:1 to 4:1, or 1:1 to 3:1, or 1:1 to 2:1, or 1.5:1 to 5:1, or 2:1 to 5:1, or 2:1 to 4:1, or 2:1 to 3:1, or about 1:1.
1.11 Any foregoing composition, wherein the composition comprises a molar ratio of nitric acid or water-soluble nitrate salt (e.g., potassium nitrate) to stannous fluoride or stannous chloride or stannous pyrophosphate of at least 0.3:1, e.g., 0.3:1 to 20:1, or 0.5:1 to 20:1, or 1:1 to 20:1, or 1:1 to 15:1, or 1:1 to 10:1, or 1:1 to 5:1 or 1:1 to 3:1, or about 1:1.
1.12 Any foregoing composition, wherein the composition comprises from 0.1 to 2% stannous fluoride, stannous chloride or stannous pyrophosphate, or combinations thereof, by weight of the composition, e.g., 0.1 to 1%, or 0.25 to 0.75%, or about 0.45%.
1.13 Any foregoing composition, wherein the composition comprises from 0.1 to 5% of the nitric acid or water-soluble nitrate salt (e.g., potassium nitrate), by weight of the composition, e.g., 0.1 to 2%, or 0.1 to 1%, or 0.1 to 0.5%, or 0.2 to 0.4%, or about 0.3%.
1.14 Any foregoing composition, wherein the composition comprises from 0.1 to 5% of the alkali metal polyphosphate salt (e.g., tetrasodium pyrophosphate or sodium tripolyphosphate), by weight of the composition, e.g., 0.8 to 5%, or 0.8 to 4%, or 0.8 to 3%, or 0.8 to 2%, or 0.8 to 1.0%, or about 0.8%.
1.15 Any foregoing composition, wherein the composition comprises at least 20% water by weight of the composition, e.g., at least 30%, or at least 40%, or at least 50%, or at least 60% or at least 65%, up to 95% water, by weight of the composition.
1.16 Any foregoing composition wherein the composition comprises 70% to 95% water, by weight of the composition, e.g., from 75% to 95%, or from 75% to 90%, or from 75% to 85%, or from 75% to 80%; or wherein the composition comprises from 10% to 50% water, by weight of the composition, e.g., 10% to 40%, or 10% to 30%.
1.17 Any foregoing composition, wherein the composition comprises one or more humectants (e.g., glycerin, sorbitol, propylene glycol, or a mixture thereof) in a net amount of 5% to 70% by weight of the composition, e.g., from 5% to 25% by weight of the composition, or from 10% to 25%, or from 15% to 25%, or about 20%, or from 30 to 70%, or from 35 to 60%, or from 40 to 60%, by weight of the composition.
1.18 Any foregoing composition, wherein the composition is a single phase, i.e., it does not form two phases on standing.
1.19 Any foregoing composition, wherein the composition is a clear (e.g., not opaque or turbid) solution (e.g., not a suspension).
1.20 Any foregoing composition, wherein the composition is physically and chemically stable, for example, wherein no color change or precipitation occurs on storage at ambient conditions for 3 months or more (e.g., 6 months or more, or 1 year or more).
1.21 Composition 1.20, wherein the stannous ion concentration is substantially stable for at least three months on storage, e.g., the concentration of stannous ion is at least 80% of the original concentration, or at least 85%, or at least 90%.
1.22 Any foregoing composition, wherein the composition has a pH of between 5 and 9, or a pH between 6 and 8, or a pH between 6.5 and 7.5, or a pH between 6.9 and 7.1, or a pH of about 7.
1.23 Any foregoing composition, wherein the composition comprises less than 10% of any hydrophobic liquid or mixture of hydrophobic liquids (e.g., alkyl fatty acid esters (e.g., isopropyl myristate), vegetable oils, mineral oils, or combinations thereof), by weight of the composition, for example, less than 5% by weight or less than 3% by weight or less than 1% by weight, of such hydrophobic liquids.
1.24 Any foregoing composition, wherein the composition is free or substantially free of any hydrophobic liquid or mixture of hydrophobic liquids (e.g., less than 0.1% by weight of the composition).
1.25 Any foregoing composition, further comprising a nonionic surfactant, e.g., a hydrophilic nonionic surfactant.
1.26 Composition 1.25, wherein the nonionic surfactant is a copolymer of ethylene oxide and propylene oxide, for example, a block copolymer (e.g., a triblock copolymer).
1.27 Composition 1.26, wherein the nonionic surfactant is a poloxamer, e.g., a triblock copolymer having a hydrophobic polypropylene glycol block flanked by hydrophilic polyethylene glycol blocks.
1.28 Composition 1.27, wherein the poloxamer has a polyethylene glycol block length of about 75 to 125 units (e.g., about 100-101), and a polypropylene block length of about 25 to 75 units (e.g., about 55-56), for example, poloxamer 407 or Pluronic F127.
1.29 Any foregoing composition, comprising a nonionic surfactant in an amount of 0.01 to 5.0%, by weight of the composition, e.g., 0.1 to 1.0%, 0.2 to 0.7%, 0.3 to 0.5%, about 0.4%.
1.30 Any foregoing composition, further comprising an anionic surfactant, e.g., selected from sodium laurel ether sulfate (SLES), sodium lauryl sulfate, and ammonium lauryl sulfate.
1.31 Any foregoing composition wherein the composition further comprises one or more of a thickener., a buffer, a sweetener, a flavorant, a pigment, a dye, an anti-caries agent, an anti-bacterial agent, a whitening agent, a desensitizing agent, a preservative, or a mixture thereof.
1.32 Any foregoing composition wherein the composition further comprises an additional fluoride ion source.
1.33 Composition 1.32, wherein the additional fluoride ion source is selected from sodium fluoride, potassium fluoride, sodium monofluorophosphate, sodium fluorosilicate, ammonium fluorosilicate., amine fluoride (e.g., N′-octadecyltrimethylendiamine-N,N,N′-tris(2-ethanol)-dihydrofluoride), ammonium fluoride, titanium fluoride, hexafluorosulfate, or a mixture thereof.
1.34 Any foregoing composition wherein the composition comprises a whitening agent.
1.35 Any foregoing composition wherein the composition comprises a whitening agent, wherein the whitening agent is hydrogen peroxide.
1.36 Any foregoing composition wherein the composition further comprises a desensitizing agent selected from potassium chloride, strontium chloride, or a mixture thereof.
1.37 Any foregoing composition wherein the composition is a mouthwash.
1.38 Any foregoing composition wherein the composition is a dentifrice (e.g., a toothpaste or a tooth gel).
1.39 Any foregoing composition, wherein the composition is free of abrasives (e.g., the composition is free of silicas).
1.40 Any foregoing composition, wherein the composition comprises abrasive (e.g. silicas) in an amount of 1-30% by weight of the composition, e.g., 10-30%, or 20-25%.
1.41 Any foregoing composition wherein the amino acid is a basic amino acid., and wherein the basic amino acid is selected from the following: arginine, lysine, serine, citrullene, ornithine, creatine, histidine, diaminobutanoic acid, diaminoproprionic acid, and combinations thereof (e.g., and salts thereof) (e.g., from 1-5% by wt.) (e.g., about 1-3% by wt.) (e.g., 5-15% by wt) (e.g., about 1.3%) (e.g., about 1.5%).
1.42 The composition of 1.41, wherein the basic amino acid is arginine (e.g., in free or salt form) (e.g., L-arginine).
1.43 The composition of 1.42, wherein the amount of arginine is from 1-15% by wt of the oral care composition. (e.g., from 1-5% by wt.) (e.g., about 1-3% by wt.) (e.g., 5-15% by wt) (e.g., about 1.3%) (e.g., about 1.5%).
1.44 Any foregoing composition further comprising an additional stannous ion source.
1.45 Any of the foregoing compositions, wherein the composition is effective upon application to the oral cavity, e.g., by rinsing, optionally in conjunction with brushing, to (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing and/or malodor producing bacteria, (viii) treat, relieve or reduce dry mouth, (ix) clean the teeth and oral cavity, (x) whiten the teeth, (xi) reduce tartar build-up, (xii) reduce or prevent oral malodor, and/or (xiii) promote systemic health, including cardiovascular health, e.g., by reducing potential for systemic infection via the oral tissues.
1.46 Any foregoing composition, wherein the composition has enhanced stannous ion stability (e.g., compared to a composition comprising stannous fluoride or stannous chloride without both a nitrate ion source and a polyphosphate).
1.47 Any foregoing composition, wherein the composition is packaged in a container comprising a single storage compartment, which compartment comprises the composition, and a closure (e.g., a screw-top closure) which seals the compartment.
1.48 Any of the foregoing compositions further comprising 0.001 to 0.025% by weight of charcoal (e.g., activated charcoal); wherein the composition is formulated as a dentifrice (e.g., toothpaste or tooth gel).
1.49 The composition of 1.48, wherein the composition comprises 0.001 to 0.020% by weight of charcoal, e.g., 0.001 to 0.015%, or 0.001 to 0.012%, or 0.005 to 0.010%, or 0.005 to 0.009%, or 0.005 to 0.0085%, by weight of charcoal.
1.50 Any of compositions 1.48 or 1.49, wherein the composition comprises 0.005 to 0.008% or 0.006 to 0.008% by weight of charcoal.
1.51 Any of compositions 1.48 to 1.50, wherein the composition comprises 0.007 to 0.008% by weight of charcoal.
1.52 Any of compositions 1.48 to 1.51, wherein the composition comprises about 0.0075% by weight of charcoal.
1.53 The composition of any of 1.48-1.52, wherein the charcoal is activated charcoal.
1.54 The composition of any of 1.48-1.53, wherein the composition has a light transmittance of at least 0.001% measured on a 10 mm-thick vertical sample, e.g., at least 0.01%, or at least 0.1%, or at least 0.2%, or 0.05% to 1%, or 0.1% to 1%, or 0.2% to 0.5%, or about 0.25%.
1.55 Any foregoing compositions, wherein the composition comprises stannous fluoride (e.g., where stannous fluoride is the only source of stannous in the composition).
1.56 Any of the preceding compositions, wherein the composition comprises stannous chloride (e.g., where stannous chloride is the only source of stannous in the composition).
1.57 Any of the preceding compositions, wherein the composition comprises stannous pyrophosphate (e.g., where stannous pyrophosphate is the only source of stannous in the composition).
1.58 Any of the preceding compositions comprising a combination of stannous fluoride and stannous chloride or stannous fluoride and stannous pyrophosphate.
1.59 Any of compositions 1.0-1.57 comprising a combination of one or more of stannous fluoride, stannous chloride, and stannous pyrophosphate.
1.60 Any of the preceding compositions, wherein the composition comprises:
   (i) stannous fluoride or stannous chloride or stannous pyrophosphate;
   (ii) potassium nitrate;
   (iii) tetrasodium pyrophosphate;
   (iv) arginine; and
   (v) more than 10% water, by weight of the composition.
1.61 Any of the preceding compositions, wherein the composition comprises:
   (i) 0.1-2% by wt. of stannous fluoride or stannous chloride or stannous pyrophosphate;
   (ii) 0.1-2% by wt. of potassium nitrate;
   (iii) 0.8-4% by wt. of tetrasodium pyrophosphate;
   (iv) 0.1%-5% by wt. of arginine; and
   (v) more than 10% water, by weight of the composition (e.g., 10%-90% by wt.)
1.62 Any of the preceding compositions, wherein the composition comprises:
   (i) 0.1-2% by wt. of stannous fluoride;
   (ii) 0.1-2% by wt. of potassium nitrate;
   (iii) 0.8-4% by wt. of tetrasodium pyrophosphate;
   (iv) 0.1%-5% by wt. of arginine; and
   (vi) more than 10% water, by weight of the composition (e.g., 10%-90% by wt.).
1.63 Any of the preceding compositions, wherein the composition comprises:
   (i) 0.1-2% by wt. of stannous chloride;
   (ii) 0.1-2% by wt. of potassium nitrate;
   (iii) 0.8-4% by wt. of tetrasodium pyrophosphate;
   (iv) 0.1%-5% by wt. of arginine; and
   (v) more than 10% water, by weight of the composition (e.g., 10%-90% by wt.)
1.64 Any of the preceding compositions, wherein the composition comprises:
   (i) 0.1-2% by wt. of stannous pyrophosphate;
   (ii) 0.1-2% by wt. of potassium nitrate;
   (iii) 0.8-4% by wt. of tetrasodium pyrophosphate;
   (iv) 0.1%-5% by wt. of arginine; and
   (v) more than 10% water, by weight of the composition (e.g., 10%-90% by wt.).

In a second aspect, the present disclosure further provides a method (Method 1) of stabilizing stannous ion in an aqueous oral care composition comprising the steps of (1) providing an aqueous vehicle, (2) adding to the aqueous vehicle a stannous ion source, (3) adding to the aqueous vehicle a nitrate ion source, (4) adding to the aqueous vehicle a an amino acid source (e.g., a basic amino acid source) and (5) adding to the aqueous vehicle a polyphosphate ion source, wherein the final composition is a single-component high-water composition.

For example, the disclosure provides embodiments of Method 1 as follows:
1.1 Method 1, wherein the stannous ion source is a water-soluble stannous salt.
1.2 Method 1 or 1.1, wherein the stannous salt is stannous fluoride or stannous chloride or stannous pyrophosphate.
1.3 Method 1.2, wherein the stannous salt is stannous fluoride.
1.4 Method 1.2, wherein the stannous salt is stannous chloride.
1.5 Method 1.2, wherein the stannous salt is stannous pyrophosphate.
1.6 Any preceding method, wherein the nitrate ion source is nitric acid or a water-soluble nitrate salt.
1.7 Method 1.6, wherein the water-soluble nitrate salt is selected from an alkali or alkaline earth metal nitrate, or zinc nitrate, silver nitrate, or ammonium nitrate.
1.8 Method 1.6, wherein the water-soluble nitrate salt is an alkali metal nitrate salt or an alkaline earth metal nitrate salt.
1.9 Method 1.8, wherein the nitrate salt is selected from lithium nitrate, sodium nitrate, potassium nitrate, magnesium nitrate, and calcium nitrate.
1.10 Method 1.9, wherein the nitrate salt is potassium nitrate.
1.11 Any preceding method, wherein the polyphosphate ion source is a water-soluble alkali metal polyphosphate.
1.12 Method 1.11, wherein the water-soluble alkali metal polyphosphate is selected from a pyrophosphate, tripolyphosphate, tetraphosphate or hexametaphosphate.
1.13 Method 1.11, wherein the water-soluble alkali metal polyphosphate is a sodium or potassium polyphosphate.
1.14 Method 1.11, wherein the water-soluble alkali metal polyphosphate is selected from sodium pyrophosphate, potassium pyrophosphate, sodium tripolyphosphate and potassium tripolyphosphate.
1.15 Method 1.14, wherein the sodium pyrophosphate salt is selected from sodium acid pyrophosphate (i.e., disodium pyrophosphate) and tetrasodium pyrophosphate.
1.16 Any preceding method, wherein the stannous salt is stannous fluoride or stannous chloride or stannous pyrophosphate, the nitrate salt is potassium nitrate, and the polyphosphate salt is tetrasodium pyrophosphate.
1.17 Any preceding method, wherein the composition is formulated to have a molar ratio of polyphosphate source (e.g., tetrasodium pyrophosphate or sodium tripolyphosphate) to stannous source (e.g., stannous fluoride, stannous chloride, or stannous pyrophosphate) of at least 1:1, e.g., 1:1 to 5:1, or 1:1 to 4:1, or 1:1 to 3:1, or 1:1 to 2:1, or 1.5:1 to 5:1, or 2:1 to 5:1, or 2:1 to 4:1, or 2:1 to 3:1, or about 1:1.
1.18 Any preceding method, wherein the composition is formulated to have a molar ratio of nitric acid or nitrate source (e.g., potassium nitrate) to stannous source (e.g., stannous fluoride, stannous chloride, or stannous pyrophosphate) of at least 0.3:1, e.g., 0.3:1 to 20:1, or 0.5:1 to 20:1, or 1:1 to 20:1, or 1:1 to 15:1, or 1:1 to 10:1, or 1:1 to 5:1 or 1:1 to 3:1, or about 1:1.
1.19 Any preceding method, wherein the composition is formulated to comprise from 0.1 to 2% stannous ion source (e.g., stannous fluoride, stannous chloride, or stannous pyrophosphate), by weight of the composition, e.g., 0.1 to 1%, or 0.25 to 0.75%, or about 0.45%.
1.20 Any preceding method, wherein the composition is formulated to comprise from 0.1 to 5% of nitric acid or nitrate ion source (e.g., potassium nitrate), by weight of the composition, e.g., 0.1 to 2%, or 0.1 to 1%, or 0.1 to 0.5%, or 0.2 to 0.4%, or about 0.3%.
1.21 Any preceding method, wherein the composition is formulated to comprise from 0.1 to 5% of polyphosphate ion source (e.g., tetrasodium pyrophosphate), by weight of the composition, e.g., 0.8 to 5%, or 0.8 to 4%, or 0.8 to 3%, or 0.8 to 2%, or 0.8 to 1.0%, or about 0.8%.
1.22 Any preceding method, wherein the amino acid is a basic amino acid, and wherein the basic amino acid is selected from the following: arginine, lysine, serine, citrullene, ornithine, creatine, histidine, diaminobutanoic acid, diaminoproprionic acid, and combinations thereof (e.g., and salts thereof) (e.g., from 1-5% by wt.) (e.g., about 1-3% by wt.) (e.g., 5-15% by wt) (e.g., about 1.3%) (e.g., about 1.5%).
1.23 The method of 1.22, wherein the basic amino acid is arginine (e.g., in free or salt form) (e.g., L-arginine).
1.24 The method of 1.23, wherein the amount of arginine is from 1-15% by wt of the oral care composition. (e.g., from 1-5% by wt.) (e.g., about 1-3% by wt.) (e.g., 5-15% by wt) (e.g., about 1.3%) (e.g., about 1.5%).
1.25 Any preceding method, wherein the aqueous vehicle comprises water and optionally one or more humectants (e.g., glycerin, sorbitol, propylene glycol, or a mixture thereof).
1.26 Any preceding method, wherein the composition is formulated to comprise from 10% to 95% water, by weight of the composition, e.g., from 20 to 95%, or from 30 to 95%, or from 40 to 95%, or from 50 to 95%, or from 60 to 95% or from 65 to 95%, by weight of the composition.
1.27 Any preceding method, wherein the composition is formulated to comprise 70% to 95% water, by weight of the composition, e.g., from 75% to 95%, or from 75% to 90%, or from 75% to 85%, or from 75% to 80%; or wherein the composition is formulated to comprise from 10% to 50% water, by weight of the composition, e.g., 10% to 40%, or 10% to 30%.
1.28 Any preceding method, wherein the composition is formulated to comprise one or more humectants (e.g., glycerin, sorbitol, propylene glycol, or a mixture thereof) in a net amount of 5 to 75% by weight of the composition, e.g., from 5% to 25% by weight of the composition, or from 10% to 25%, or from 15% to 25%, or about 20%, or from 30 to 70%, or from 35 to 60%, or from 40 to 60%, by weight of the composition.
1.29 Any preceding method, wherein the composition is formulated as a single phase, i.e., it does not form two phases on standing.
1.30 Any preceding method, wherein the composition is formulated as a clear (e.g., not opaque or turbid) solution (e.g., not a suspension).
1.31 Any preceding method, wherein the composition is physically and chemically stable, for example, wherein no color change or precipitation occurs on storage at ambient conditions for 3 months or more (e.g., 6 months or more, or 1 year or more).
1.32 Method 1.31, wherein the stannous ion concentration is substantially stable for at least three months on storage. e.g., the concentration of stannous ion is at least 80% of the original concentration, or at least 85%, or at least 90%.
1.33 Any preceding method, wherein the composition has a pH of between 5 and 9, or a pH between 6 and 8, or a pH between 6.5 and 7.5, or a pH between 6.9 and 7.1, or a pH of about 7.
1.34 Any preceding method, wherein the composition is formulated to comprise less than 10% of any hydrophobic liquid or mixture of hydrophobic liquids (e.g., alkyl fatty acid esters (e.g., isopropyl myristate), vegetable oils, mineral oils, or combinations thereof), by weight of the composition, for example, less than 5% by weight or less than 3% by weight or less than 1% by weight, of such hydrophobic liquids.
1.35 Any preceding method, wherein the composition is formulated to be free or substantially free of any hydrophobic liquid or mixture of hydrophobic liquids (e.g., less than 0.1% by weight of the composition), i.e., the method does not comprise any step of adding any hydrophobic liquid to the aqueous vehicle.
1.36 Any preceding method, wherein the composition is formulated to comprise a nonionic surfactant, e.g., a hydrophilic nonionic surfactant, i.e., the method further comprises the step (5) of adding a nonionic surfactant to the aqueous vehicle.
1.37 Method 1.36, wherein the nonionic surfactant is a copolymer of ethylene oxide and propylene oxide, for example, a block copolymer (e.g., a triblock copolymer).
1.38 Method 1.36, wherein the nonionic surfactant is a poloxamer, e.g., a triblock copolymer having a hydrophobic polypropylene glycol block flanked by hydrophilic polyethylene glycol blocks.
1.39 Method 1.38, wherein the poloxamer has a polyethylene glycol block length of about 75 to 125 units (e.g., about 100-101), and a polypropylene block length of about 25 to 75 units (e.g., about 55-56), for example, poloxamer 407 or Pluronic F127.
1.40 Any of methods 1.36-1.39, wherein the composition is formulated to comprise the nonionic surfactant in an amount of 0.01 to 5.0%, by weight of the composition, e.g., 0.1 to 1.0%, 0.2 to 0.7%, 0.3 to 0.5%, about 0.4% 1.41 Any preceding method, wherein the composition is a mouthwash.
1.42 Any preceding method, wherein the composition is a dentifrice (e.g., a toothpaste or a tooth gel).
1.43 Any preceding method, wherein the composition is formulated to comprise abrasive (e.g. silicas) in an amount of 1-30% by weight of the composition, e.g., 10-30%, or 20-25%.
1.44 Any preceding method, wherein the composition is formulated to be free of abrasives (e.g., the composition is formulated to be free of silicas).
1.45 Any preceding method, wherein step (1) occurs first and steps (2)-(5) occur in any order.
1.46 Any preceding method, further comprising a final step (6) of packaging the composition in a container comprising a single storage compartment, which compartment comprises the composition, and a closure (e.g., a screw-top closure) which seals the compartment.
1.47 Any preceding method, wherein the method results in a composition according to Composition 1.0 et seq.

In a third aspect, the present disclosure provides an oral care package comprising a composition according to Composition 1.0 et seq, wherein the package comprises a container comprising a single storage compartment, which compartment contains the composition, and a closure (e.g., a screw-top closure) which seals the compartment.

In a fourth aspect, the present disclosure provides a method of treatment or prevention of gingivitis, plaque, dental caries, and/or dental hypersensitivity, the method comprising the application to the oral cavity of a person in need thereof, of a composition according to the invention (e.g., Composition 1.0 et seq.), e.g., by brushing, for example, one or more times per day.

Alternatively, the present disclosure provides Composition 1.0, et seq., for use in the treatment or prevention of gingivitis, plaque, dental caries, and/or dental hypersensitivity.

The methods of the fourth aspect comprise applying any of the compositions as described herein to the teeth, e.g., by brushing, gargling or rinsing, or otherwise administering the compositions to the oral cavity of a subject in need thereof. The compositions can be administered regularly, such as, for example, one or more times per day (e.g., twice per day). In various embodiments, administering the compositions of the present disclosure to teeth may provide one or more of the following specific benefits: (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing and/or malodor producing bacteria, (viii) treat, relieve or reduce dry mouth, (ix) clean the teeth and oral cavity, (x) whiten the teeth, (xi) reduce tartar build-up, (xii) reduce or prevent oral malodor, and/or (xiii) promote systemic health, including cardiovascular health, e.g., by reducing potential for systemic infection via the oral tissues.

In a fifth aspect, the present disclosure provides a method (Method 2.0) of treating or reducing systemic blood pressure in a subject (e.g., patient) in need thereof the method comprising the application to the oral cavity of a person in need thereof (e.g., wherein the person has elevated blood pressure or is at risk for elevated blood pressure), of a composition according to the invention (e.g., and of Composition 1.0 et seq.), e.g., by brushing, for example, one or more times per day. For example, the method comprises administering a single-component oral care composition comprising:
(i) stannous fluoride or stannous chloride or stannous pyrophosphate;
(ii) nitric acid or a water-soluble nitrate salt (e.g., potassium nitrate);
(iii) a water-soluble alkali metal polyphosphate (e.g., sodium or potassium pyrophosphate or tripolyphosphate);
(iv) an amino acid (e.g., a basic amino acid) (e.g., arginine) (e.g., in free or salt form); and
(v) more than 10% water, by weight of the composition.

For example, the disclosure provides embodiments of Method 2.0 as follows:
2.1 Method 2.0, wherein the water-soluble nitrate salt is selected from an alkali or alkaline earth metal nitrate, or zinc nitrate, silver nitrate, or ammonium nitrate.
2.2 Method 2.1, wherein the water-soluble nitrate salt is an alkali metal nitrate salt or an alkaline earth metal nitrate salt.
2.3 Method 2.2, wherein the nitrate salt is selected from lithium nitrate, sodium nitrate, potassium nitrate, magnesium nitrate, and calcium nitrate.
2.4 Method 2.3, wherein the nitrate salt is potassium nitrate.
2.5 Any foregoing method, wherein the water-soluble alkali metal polyphosphate is selected from a pyrophosphate, tripolyphosphate, tetraphosphate or hexametaphosphate.
2.6 Any foregoing method, wherein the water-soluble alkali metal polyphosphate is a sodium or potassium polyphosphate.
2.7 Any foregoing method, wherein the water-soluble alkali metal polyphosphate is selected from sodium pyrophosphate, potassium pyrophosphate, sodium tripolyphosphate and potassium tripolyphosphate.
2.8 Method 2.7, wherein the sodium pyrophosphate salt is selected from sodium acid pyrophosphate (i.e., disodium pyrophosphate) and tetrasodium pyrophosphate.
2.9 Any foregoing method, wherein the water-soluble nitrate salt is potassium nitrate and the water-soluble alkali metal polyphosphate salt is tetrasodium pyrophosphate.
2.10 Any foregoing method, wherein the composition comprises a molar ratio of alkali metal polyphosphate (e.g., tetrasodium pyrophosphate) to stannous fluoride or stannous chloride or stannous pyrophosphate of at least 1:1, e.g., 1:1 to 5:1, or 1:1 to 4:1, or 1:1 to 3:1, or 1:1 to 2:1, or 1.5:1 to 5:1, or 2:1 to 5:1, or 2:1 to 4:1, or 2:1 to 3:1, or about 1:1.
2.11 Any foregoing method, wherein the composition comprises a molar ratio of nitric acid or water-soluble nitrate salt (e.g., potassium nitrate) to stannous fluoride or stannous chloride or stannous pyrophosphate of at least 0.3:1, e.g., 0.3:1 to 20:1, or 0.5:1 to 20:1, or 1:1 to 20:1, or 1:1 to 15:1, or 1:1 to 10:1, or 1:1 to 5:1 or 1:1 to 3:1, or about 1:1.
2.12 Any foregoing method, wherein the composition comprises from 0.1 to 2% stannous fluoride, stannous chloride or stannous pyrophosphate, or combinations thereof, by weight of the composition, e.g., 0.1 to 1%, or 0.25 to 0.75%, or about 0.45%.
2.13 Any foregoing method, wherein the composition comprises from 0.1 to 5% of the nitric acid or water-soluble nitrate salt (e.g., potassium nitrate), by weight of the composition, e.g., 0.1 to 2%, or 0.1 to 1%, or 0.1 to 0.5%, or 0.2 to 0.4%, or about 0.3%.
2.14 Any foregoing method, wherein the composition comprises from 0.1 to 5% of the alkali metal polyphosphate salt (e.g., tetrasodium pyrophosphate or sodium tripolyphosphate), by weight of the composition, e.g., 0.8 to 5%, or 0.8 to 4%, or 0.8 to 3%, or 0.8 to 2%, or 0.8 to 1.0%, or about 0.8%.
2.15 Any foregoing method, wherein the composition comprises at least 20% water by weight of the composition, e.g., at least 30%, or at least 40%, or at least 50%, or at least 60% or at least 65%, up to 95% water, by weight of the composition.
2.16 Any foregoing method wherein the composition comprises 70% to 95% water, by weight of the composition, e.g., from 75% to 95%, or from 75% to 90%, or from 75% to 85%, or from 75% to 80%; or wherein the composition comprises from 10% to 50% water, by weight of the composition. e.g., 10% to 40%, or 10% to 30%.
2.17 Any foregoing method, wherein the composition comprises one or more humectants (e.g., glycerin, sorbitol, propylene glycol, or a mixture thereof) in a net amount of 5% to 70% by weight of the composition, e.g., from 5% to 25% by weight of the composition, or from 10% to 25%, or from 15% to 25%, or about 20%, or from 30 to 70%, or from 35 to 60%, or from 40 to 60%, by weight of the composition.
2.18 Any foregoing method, wherein the composition is a single phase, i.e., it does not form two phases on standing.
2.19 Any foregoing method, wherein the composition is a clear (e.g., not opaque or turbid) solution (e.g., not a suspension).
2.20 Any foregoing method, wherein the composition is physically and chemically stable, for example, wherein no color change or precipitation occurs on storage at ambient conditions for 3 months or more (e.g., 6 months or more, or 1 year or more).
2.21 Method 2.20, wherein the stannous ion concentration is substantially stable for at least three months on storage, e.g., the concentration of stannous ion is at least 80% of the original concentration, or at least 85%, or at least 90%.
2.22 Any foregoing method, wherein the composition has a pH of between 5 and 9, or a pH between 6 and 8, or a pH between 6.5 and 7.5, or a pH between 6.9 and 7.1, or a pH of about 7.
2.23 Any foregoing method, wherein the composition comprises less than 10% of any hydrophobic liquid or mixture of hydrophobic liquids (e.g., alkyl fatty acid esters (e.g., isopropyl myristate), vegetable oils, mineral oils, or combinations thereof), by weight of the composition, for example, less than 5% by weight or less than 3% by weight or less than 1% by weight, of such hydrophobic liquids.
2.24 Any foregoing method, wherein the composition is free or substantially free of any hydrophobic liquid or mixture of hydrophobic liquids (e.g., less than 0.1% by weight of the composition).
2.25 Any foregoing method, further comprising a nonionic surfactant, e.g., a hydrophilic nonionic surfactant.
2.26 Method 2.25, wherein the nonionic surfactant is a copolymer of ethylene oxide and propylene oxide, for example, a block copolymer (e.g., a triblock copolymer).
2.27 Method 2.26, wherein the nonionic surfactant is a poloxamer, e.g., a triblock copolymer having a hydrophobic polypropylene glycol block flanked by hydrophilic polyethylene glycol blocks.
2.28 Method 2.27, wherein the poloxamer has a polyethylene glycol block length of about 75 to 125 units (e.g., about 100-101), and a polypropylene block length of about 25 to 75 units (e.g., about 55-56), for example, poloxamer 407 or Pluronic F127.
2.29 Any foregoing method, comprising a nonionic surfactant in an amount of 0.01 to 5.0%, by weight of the composition, e.g., 0.1 to 1.0%, 0.2 to 0.7%, 0.3 to 0.5%, about 0.4%.
2.30 Any foregoing method, further comprising an anionic surfactant, e.g., selected from sodium laurel ether sulfate (SLES), sodium lauryl sulfate, and ammonium lauryl sulfate.
2.31 Any foregoing method wherein the composition further comprises one or more of a thickener, a buffer, a sweetener, a flavorant, a pigment, a dye, an anti-caries agent, an anti-bacterial agent, a whitening agent, a desensitizing agent, a preservative, or a mixture thereof.
2.32 Any foregoing method wherein the composition further comprises an additional fluoride ion source.
2.33 Method 2.32, wherein the additional fluoride ion source is selected from sodium fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride), ammonium fluoride, titanium fluoride, hexafluorosulfate, or a mixture thereof.
2.34 Any foregoing method wherein the composition comprises a whitening agent.
2.35 Any foregoing method wherein the composition comprises a whitening agent, wherein the whitening agent is hydrogen peroxide.
2.36 Any foregoing method wherein the composition further comprises a desensitizing agent selected from potassium chloride, strontium chloride, or a mixture thereof.
2.37 Any foregoing method wherein the composition is a mouthwash.
2.38 Any foregoing method wherein the composition is a dentifrice (e.g., a toothpaste or a tooth gel).
2.39 Any foregoing method, wherein the composition is free of abrasives (e.g., the composition is free of silicas).
2.40 Any foregoing method, wherein the composition comprises abrasive (e.g., silicas) in an amount of 1-30% by weight of the composition, e.g., 10-30%, or 20-25%.
2.41 Any foregoing method wherein the amino acid is a basic amino acid, and wherein the basic amino acid is selected from the following: arginine, lysine, serine, citrullene, ornithine, creatine, histidine, diaminobutanoic acid, diaminoproprionic acid, and combinations thereof (e.g., and salts thereof) (e.g., from 1-5% by wt.) (e.g., about 1-3% by wt.) (e.g., 5-15% by wt.) (e.g., about 1.3%) (e.g., about 1.5%).
2.42 The method of 2.41, wherein the basic amino acid is arginine (e.g., in free or salt form) (e.g., L-arginine).
2.43 The method of 1.42, wherein the amount of arginine is from 1-15% by wt. of the oral care composition. (e.g., from 1-5% by wt.) (e.g., about 1-3% by wt.) (e.g., 5-15% by wt.) (e.g., about 1.3%) (e.g., about 1.5%).
2.44 Any foregoing method further comprising an additional stannous ion source.
2.45 Any of the foregoing method, wherein the composition is effective upon application to the oral cavity, e.g., by rinsing, optionally in conjunction with brushing, to (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing and/or malodor producing bacteria, (viii) treat, relieve or reduce dry mouth, (ix) clean the teeth and oral cavity, (x) whiten the teeth, (xi) reduce tartar build-up, (xii) reduce or prevent oral malodor, and/or (xiii) promote systemic health, including cardiovascular health, e.g., by reducing potential for systemic infection via the oral tissues.
2.46 Any foregoing method, wherein the composition has enhanced stannous ion stability (e.g., compared to a composition comprising stannous fluoride or stannous chloride without both a nitrate ion source and a polyphosphate).
2.47 Any foregoing method, wherein the composition is packaged in a container comprising a single storage compartment, which compartment comprises the composition, and a closure (e.g., a screw-top closure) which seals the compartment.
2.48 Any of the foregoing method further comprising 0.001 to 0.025% by weight of charcoal (e.g., activated charcoal); wherein the composition is formulated as a dentifrice (e.g., toothpaste or tooth gel).
2.49 The method of 2.48, wherein the composition comprises 0.001 to 0.020% by weight of charcoal, e.g., 0.001 to 0.015%, or 0.001 to 0.012%, or 0.005 to 0.010%, or 0.005 to 0.009%, or 0.005 to 0.0085%, by weight of charcoal.
2.50 Any of methods 2.48 or 2.49, wherein the composition comprises 0.005 to 0.008% or 0.006 to 0.008% by weight of charcoal.
2.51 Any of method 2.48 to 2.50, wherein the composition comprises 0.007 to 0.008% by weight of charcoal.
2.52 Any of method 2.48 to 2.51, wherein the composition comprises about 0.0075% by weight of charcoal.
2.53 The method of any of 2.48-2.52, wherein the charcoal is activated charcoal.
2.54 The method of any of 2.48-2.53, wherein the composition has a light transmittance of at least 0.001% measured on a 10 mm-thick vertical sample, e.g., at least 0.01%, or at least 0.1%, or at least 0.2%, or 0.05% to 1%, or 0.1% to 1%, or 0.2% to 0.5%, or about 0.25%.
2.55 Any foregoing methods, wherein the composition comprises stannous fluoride (e.g., where stannous fluoride is the only source of stannous in the composition).
2.56 Any of the preceding methods, wherein the composition comprises stannous chloride (e.g., where stannous chloride is the only source of stannous in the composition).
2.57 Any of the preceding methods, wherein the composition comprises stannous pyrophosphate (e.g., where stannous pyrophosphate is the only source of stannous in the composition).
2.58 Any of the preceding methods comprising a combination of stannous fluoride and stannous chloride or stannous fluoride and stannous pyrophosphate.
2.59 Any of c methods 2.0-2.57 comprising a combination of one or more of stannous fluoride, stannous chloride, and stannous pyrophosphate.
2.60 Any of the preceding methods, wherein the composition comprises:
   (vi) stannous fluoride or stannous chloride or stannous pyrophosphate;
   (vii) potassium nitrate;
   (viii) tetrasodium pyrophosphate;
   (ix) arginine; and
   (x) more than 10% water, by weight of the composition.
2.61 Any of the preceding methods, wherein the composition comprises:
   (vii) 0.1-2% by wt. of stannous fluoride or stannous chloride or stannous pyrophosphate;
   (viii) 0.1-2% by wt. of potassium nitrate;
   (ix) 0.8-4% by wt. of tetrasodium pyrophosphate;
   (x) 0.1%-5% by wt. of arginine; and
   (xi) more than 10% water, by weight of the composition (e.g., 10%-90% by wt.)

In a sixth aspect, the present disclosure provides a method of treating or reducing systemic blood pressure in a subject (e.g., patient) in need thereof the method comprising the application to the oral cavity of a person in need thereof, of a composition according to the disclosure (e.g., any of Composition 1.0 et seq.), e.g., by brushing, for example, one or more times per day. In one aspect, the subject in need thereof has elevated blood pressure and/or is at risk for elevated blood pressure and wherein the administration of the composition, e.g., any of Composition 1.0 et seq., lowers or reduces the blood pressure relative to what it was prior to administration of the composition. In one aspect, administration of the composition, e.g., any of Composition 1.0 et seq., increases the presence of one or more bacteria selected from: Actinomyces naeslundii, Actinomyces odontolyticus, Actinomyces oris, Actinomyces viscosus, Bacillus brevis, Capnocytophaga sputigena, Corynebacterium durum, Corynebacterium matruchotii, Eikenella corrodens, Granulicatella adiacens, Haemophilus parainfluenzae, Haemophilus segnis, Microbacterium oxydans, Neisseria flavescens, Neisseria sicca, Neisseria subflava, Prevotella melaninogenica, Prevotella salivae, Priopionibacterium acnes, Rothia denticariosa, Rothia mucilaginosa, Staphylococcus epidermidis, Staphylococcus hemolyticus, Selenomonas noxia, Veillonella dispar, Veillonella parvula, Veillonella atypica, and combinations thereof

In a seventh aspect, the present disclosure provides a method of treating or reducing systemic blood pressure in a subject (e.g., patient) in need thereof the method comprising the application to the oral cavity of a person in need thereof, of a composition according to the disclosure (e.g., any of Composition 1.0 et seq.), e.g., by brushing, for example, one or more times per day. In this aspect, the method further comprises administering any of Composition 1.0 et seq in order to increase the presence of one or more bacteria selected from: Actinomyces naeslundii, Actinomyces odontolyticus, Actinomyces oris, Actinomyces viscosus, Bacillus brevis, Capnocytophaga sputigena, Corynebacterium durum, Corynebacterium matruchotii, Eikenella corrodens, Granulicatella adiacens, Haemophilus parainfluenzae, Haemophilus segnis, Microbacterium oxydans, Neisseria flavescens, Neisseria sicca, Neisseria subflava, Prevotella melaninogenica, Prevotella salivae, Priopionibacterium acnes, Rothia denticariosa, Rothia mucilaginosa, Staphylococcus epidermidis, Staphylococcus hemolyticus, Selenomonas noxia, Veillonella dispar, Veillonella parvula, Veillonella atypica, and combinations thereof. In certain aspect aspects, the method can further comprise administering a Composition of 1.0 et seq to deliver substrates to oral bacteria, where the substrates are designed to target and promote oral bacteria capable of metabolizing nitrate in a subject (e.g., patient) in need thereof. In one aspect, the subject (e.g., patient) in need thereof, has elevated blood pressure and/or is at risk for elevated blood pressure and the administration of the composition, e.g., any of Composition 1.0, et seq, reduces systemic blood pressure (e.g., relative to the subject's systemic blood pressure measurement prior to administration of the composition).

In one aspect, a composition according to the disclosure, e.g., any of Composition 1.0 et seq, can be administered to a subject (e.g., patient) in need thereof in order to increase the presence of a bacteria selected from: Prevotella melaninogenica, Veillonella dispar, Haemophilus parainfluenzae, Neisseria subflava, Veillonella parvula, Rothia mucilaginosa Rothia dentocariosa, and Actinomyces viscosus. In one aspect, the subject (e.g., patient) in need thereof, has elevated blood pressure and/or is at risk for elevated blood pressure and the administration of the composition, e.g., any of Composition 1.0, et seq, reduces systemic blood pressure (e.g., relative to the subject's systemic blood pressure measurement prior to administration of the composition).

In one aspect, a composition according to the disclosure, e.g., any of Composition 1.0 et seq., can be administered to a subject (e.g., a patient) in need thereof in order to increase the presence of Neisseria subflava. In one aspect, the subject (e.g., patient) in need thereof, has elevated blood pressure and/or is at risk for elevated blood pressure and the administration of the composition, e.g., any of Composition 1.0, et seq, reduces systemic blood pressure (e.g., relative to the subject's systemic blood pressure measurement prior to administration of the composition).

In an eight aspect, the present disclosure provides a method of treating or reducing systemic blood pressure in a subject (e.g., patient) in need thereof the method comprising the application to the oral cavity of a person in need thereof, of a composition according to the invention (e.g., any of Composition 1.0 et seq.), wherein the application of the composition to the oral cavity increases the amount of nitric oxide in the subject's blood plasma.

As used herein, an “oral care composition” refers to a composition for which the intended use includes oral care, oral hygiene, and/or oral appearance, or for which the intended method of use comprises administration to the oral cavity, and refers to compositions that are palatable and safe for topical administration to the oral cavity, and for providing a benefit to the teeth and/or oral cavity. The term “oral care composition” thus specifically excludes compositions which are highly toxic, unpalatable, or otherwise unsuitable for administration to the oral cavity. In some embodiments, an oral care composition is not intentionally swallowed, but is rather retained in the oral cavity for a time sufficient to affect the intended utility. The oral care compositions as disclosed herein may be used in nonhuman mammals such as companion animals (e.g., dogs and cats), as well as by humans. In some embodiments, the oral care compositions as disclosed herein are used by humans. Oral care compositions include, for example, dentifrice and mouthwash. In some embodiments, the disclosure provides mouthwash formulations.

As used herein, “single component” means an oral care composition comprising at most a single compositional component at any time. Thus, this is in distinction to a “dual-component” compositions, which is manufactured as two separate compositions, maintained separately until final point of use. For example, a dual component toothpaste is typically packaged in a tube containing two parallel compartments exiting via a common nozzle such that when the user extrudes the toothpaste from the package the two components mix immediately prior to application to the oral cavity. Likewise, a dual component mouthwash is typically packaged in a bottle comprising two compartments such that a measured amount of the liquid from each compartment is dispensed and mixed when the user. Dual component compositions are often used to maintain in separate components and compartments ingredients which are mutually incompatible, such that if kept in the same component they would adversely react or interfere with each other.

In contrast, a dual-phase composition, such as a mouthwash, is a single-component composition comprising two immiscible liquids which settle into two phases on standing. Such a composition has no need for separated compartments for storage because the natural tendency of the two phases to separate helps ensure that the ingredients in one phase are not maintained in intimate contact with the ingredients of the other phase. Nevertheless, when vigorously mixed, the two phases become intimately combined (such as, to form an emulsion), which may or may not separate back into the two phases on standing.

### Fluoride Ion Source

The disclosed oral care compositions, e.g., any of Composition 1.0 et seq., may further include one or more fluoride ion sources, e.g., soluble fluoride salts. A wide variety of fluoride ion-yielding materials can be employed as sources of soluble fluoride in the present compositions. Examples of suitable fluoride ion-yielding materials are found in U.S. Pat. No. 3,535,421, to Briner et al.; U.S. Pat. No. 4,885,155, to Parran, Jr. et al, and U.S. Pat. No. 3,678,154, to Widder et al., each of which are incorporated herein by reference. Representative fluoride ion sources used with the present disclosure (e.g., Composition 1.0 et seq.) include, but are not limited to, stannous fluoride, sodium fluoride, potassium fluoride, sodium monofluorophosphate, sodium fluorosilicate, ammonium fluorosilicate, amine fluoride, ammonium fluoride, and combinations thereof. In certain embodiments the fluoride ion source includes stannous fluoride, sodium fluoride, sodium monofluorophosphate as well as mixtures thereof. Where the formulation comprises calcium salts, the fluoride salts are preferably salts wherein the fluoride is covalently bound to another atom, e.g., as in sodium monofluorophosphate, rather than merely ionically bound, e.g., as in sodium fluoride.

### Surfactants

The disclosed compositions may in some embodiments contain anionic surfactants, e.g., any of Composition 1.0, et seq., for example, water-soluble salts of higher fatty acid monoglyceride monosulfates, such as the sodium salt of the monosulfated monoglyceride of hydrogenated coconut oil fatty acids such as sodium N-methyl N-cocoyl taurate, sodium cocomo-glyceride sulfate; higher alkyl sulfates, such as sodium lauryl sulfate; higher alkyl-ether sulfates, e.g., of formula CH₃(CH₂)ₘCH₂(OCH₂CH₂)ₙOSO₃X, wherein m is 6-16, e.g., 10, n is 1-6, e.g., 2, 3 or 4, and X is Na or, for example sodium laureth-2 sulfate (CH₃(CH2)₁₀CH₂(OCH₂CH₂)₂OSO₃Na); higher alkyl aryl sulfonates such as sodium dodecyl benzene sulfonate (sodium lauryl benzene sulfonate); higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate. By “higher alkyl” is meant, e.g., C₆₋₃₀ alkyl. In particular embodiments, the anionic surfactant (where present) is selected from sodium lauryl sulfate and sodium ether lauryl sulfate. When present, the anionic surfactant is present in an amount which is effective, e.g., >0.001% by weight of the formulation, but not at a concentration which would be irritating to the oral tissue, e.g., 1%, and optimal concentrations depend on the particular formulation and the particular surfactant. In one embodiment, the anionic surfactant is present at from 0.03% to 5% by weight, e.g., about 1.75% by wt.

In another embodiment, cationic surfactants useful in any of the disclosed compositions, e.g., any of Composition 1.0, et seq., can be broadly defined as derivatives of aliphatic quaternary ammonium compounds having one long alkyl chain containing 8 to 18 carbon atoms such as lauryl trimethylammonium chloride, cetyl pyridinium chloride, cetyl trimethylammonium bromide, di-isobutylphenoxyethyldimethylbenzylammonium chloride, coconut alkyltrimethylammonium nitrite, cetyl pyridinium fluoride, and mixtures thereof. Illustrative cationic surfactants are the quaternary ammonium fluorides described in U.S. Pat. No. 3,535,421, to Briner et al., herein incorporated by reference. Certain cationic surfactants can also act as germicides in the compositions.

Illustrative nonionic surfactants of any of the disclosed compositions, e.g., any of Composition 1.0, et seq., that can be used in the compositions of the disclosure can be broadly defined as compounds produced by the condensation of alkylene oxide groups (hydrophilic in nature) with an organic hydrophobic compound which may be aliphatic or alkylaromatic in nature. Examples of suitable nonionic surfactants include, but are not limited to, the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials. In a particular embodiment, the composition of the invention comprises a nonionic surfactant selected from polaxamers (e.g., polaxamer 407), polysorbates (e.g., polysorbate 20), polyoxyl hydrogenated castor oils (e.g., polyoxyl 40 hydrogenated castor oil), and mixtures thereof.

Illustrative amphoteric surfactants of any of the disclosed compositions, e.g., any of Composition 1.0, et seq., that can be used in the compositions of the disclosure include betaines (such as cocamidopropylbetaine), derivatives of aliphatic secondary and tertiary amines in which the aliphatic radical can be a straight or branched chain and wherein one of the aliphatic substituents contains about 8-18 carbon atoms and one contains an anionic water-solubilizing group (such as carboxylate, sulfonate, sulfate, phosphate or phosphonate), and mixtures of such materials.

The surfactant or mixtures of compatible surfactants can be present in the compositions of the present invention in 0.1% to 5%, in another embodiment 0.3% to 3% and in another embodiment 0.5% to 2% by weight of the total composition.

### Flavoring Agents

In another aspect, any of the disclosed compositions, e.g., any of Composition 1.0, et seq., may also include a flavoring agent. Flavoring agents which are used in the practice of the present invention include, but are not limited to, essential oils and various flavoring aldehydes, esters, alcohols, and similar materials, as well as sweeteners such as sodium saccharin. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Certain embodiments employ the oils of peppermint and spearmint.

The flavoring agent is incorporated in the oral composition at a concentration of 0.01 to 1% by weight.

### pH Adjusting Agents

In some embodiments, any of the compositions of the present disclosure, e.g., any of Composition 1.0 et seq, can contain a buffering agent. Examples of buffering agents include anhydrous carbonates such as sodium carbonate, sesquicarbonates, bicarbonates such as sodium bicarbonate, silicates, bisulfates, phosphates (e.g., monopotassium phosphate, monosodium phosphate, disodium phosphate, dipotassium phosphate, tribasic sodium phosphate, sodium tripolyphosphate, pentapotassium tripolyphosphate, phosphoric acid), citrates (e.g. citric acid, trisodium citrate dehydrate), pyrophosphates (sodium and potassium salts, e.g., tetrapotassium pyrophosphate) and combinations thereof. The amount of buffering agent is sufficient to provide a pH of about 5 to about 9, preferable about 6 to about 8, and more preferable about 7, when the composition is dissolved in water, a mouthrinse base, or a toothpaste base. Typical amounts of buffering agent are about 5% to about 35%, in one embodiment about 10% to about 30%, in another embodiment about 15% to about 25%, by weight of the total composition.

### Chelating and Anti-Calculus Agents

In one aspect, the oral care compositions of the disclosure, e.g., any of Composition 1.0 et seq., may include one or more chelating agents able to complex calcium found in the cell walls of the bacteria. Binding of this calcium weakens the bacterial cell wall and augments bacterial lysis.

Another group of agents suitable for use as chelating or anti-calculus agents in the present invention are the soluble pyrophosphates. The pyrophosphate salts used in the present compositions can be any of the alkali metal pyrophosphate salts. In certain embodiments, salts include tetra alkali metal pyrophosphate, dialkali metal diacid pyrophosphate, trialkali metal monoacid pyrophosphate and mixtures thereof, wherein the alkali metals are sodium or potassium. The salts are useful in both their hydrated and unhydrated forms. An effective amount of pyrophosphate salt useful in the present composition is generally enough to provide at least 0.1 wt. % pyrophosphate ions, e.g., 0.1 to 3 wt. %, e.g., 0.1 to 2 wt. %, e.g., 0.1 to 1 wt. %, e.g., 0.2 to 0.5 wt %. The pyrophosphates also contribute to preservation of the compositions by lowering water activity.

Suitable anticalculus agents for the disclosure (e.g., Composition 1.0 et seq) include without limitation phosphates and polyphosphates (for example pyrophosphates), polyaminopropanesulfonic acid (AMPS), hexametaphosphate salts, zinc citrate trihydrate, polypeptides, polyolefin sulfonates, polyolefin phosphates, diphosphonates. In particular embodiments, the invention includes alkali phosphate salts, i.e., salts of alkali metal hydroxides or alkaline earth hydroxides, for example, sodium, potassium or calcium salts. “Phosphate” as used herein encompasses orally acceptable mono- and polyphosphates, for example, P₁₋₆ phosphates, for example monomeric phosphates such as monobasic, dibasic or tribasic phosphate; dimeric phosphates such as pyrophosphates; and multimeric phosphates, e.g., sodium hexametaphosphate. In particular examples, the selected phosphate is selected from alkali dibasic phosphate and alkali pyrophosphate salts, e.g., selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, and mixtures of any of two or more of these. In a particular embodiment, for example the compositions comprise a mixture of tetrasodium pyrophosphate (Na₄P₂O₇), calcium pyrophosphate (Ca₂P₂O₇), and sodium phosphate dibasic (Na₂HPO₄), e.g., in amounts of ca. 3-4% of the sodium phosphate dibasic and ca. 0.2-1% of each of the pyrophosphates. In another embodiment, the compositions comprise a mixture of tetrasodium pyrophosphate (TSPP) and sodium tripolyphosphate (STPP)(Na₅P₃O₁₀), e.g., in proportions of TSPP at about 1-2% and STPP at about 7% to about 10%. Such phosphates are provided in an amount effective to reduce erosion of the enamel, to aid in cleaning the teeth, and/or to reduce tartar buildup on the teeth, for example in an amount of 2-20%, e.g., ca. 5-15%, by weight of the composition.

### Polymers

The oral care compositions of the disclosure, e.g., any of Composition 1.0 et seq., also optionally include one or more polymers, such as polyethylene glycols, polyvinyl methyl ether maleic acid copolymers, polysaccharides (e.g., cellulose derivatives, for example carboxymethyl cellulose, or polysaccharide gums, for example xanthan gum or carrageenan gum). Acidic polymers, for example polyacrylate gels, may be provided in the form of their free acids or partially or fully neutralized water soluble alkali metal (e.g., potassium and sodium) or ammonium salts. Certain embodiments include 1:4 to 4:1 copolymers of maleic anhydride or acid with another polymerizable ethylenically unsaturated monomer, for example, methyl vinyl ether (methoxyethylene) having a molecular weight (M.W.) of about 30,000 to about 1,000,000. These copolymers are available for example as Gantrez AN 139(M.W. 500,000), AN 1 19 (M.W. 250,000) and S-97 Pharmaceutical Grade (M.W. 70,000), of GAF Chemicals Corporation.

Other operative polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1 103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

The N-vinyl-2-pyrrolidione is also commonly known as polyvinylpyrrolidone or “PVP”. PVP refers to a polymer containing vinylpyrrolidone (also referred to as N-vinylpyrrnlidone and N-vinyl-2-pyrrolidinone) as a monomeric unit. The monomeric unit consists of a polar imide group, four non-polar methylene groups and a non-polar methane group. The polymers include soluble and insoluble homopolymeric PVPs. Copolymers containing PVP include vinylpyrrolidone/vinyl acetate (also known as Copolyvidone, Copolyvidonum or VP-VAc) and vinyl pyrrolidone/dimethylamino-ethylmethacrylate. Soluble PVP polymers among those useful herein are known in the art, including Povidone, Polyvidone, Polyvidonum, poly(N-vinyl-2-pyrrolidinone), poly (N-vinylbutyrolactam), poly(1-vinyl-2-pyrrolidone) and poly [1-(2-oxo-1 pyrrolidinyl)ethylene]. These PVP polymers are not substantially cross-linked. In some embodiments the polymer comprises an insoluble cross-linked homopolymer. Such polymers include crosslinked PVP (often referred to as cPVP, polyvinylpolypyrrolidone, or cross-povidone).

Suitable generally, are polymerized olefinically or ethylenically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility.

A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2 methylpropane sulfonic acid having a molecular weight of about 1,000 to about 2,000,000, described in U.S. Pat. No. 4,842,847, Jun. 27, 1989 to Zahid, incorporated herein by reference.

In preparing oral care compositions, it is sometimes necessary to add some thickening material to provide a desirable consistency or to stabilize or enhance the performance of the formulation. In certain embodiments, the thickening agents are carboxyvinyl polymers, carrageenan, xanthan, hydroxyethyl cellulose and water soluble salts of cellulose ethers such as sodium carboxymethyl cellulose and sodium carboxymethyl hydroxyethyl cellulose. Natural gums such as karaya, gum arabic, and gum tragacanth can also be incorporated. Colloidal magnesium aluminum silicate or finely divided silica can be used as component of the thickening composition to further improve the composition's texture. In certain embodiments, thickening agents in an amount of about 0.5% to about 5.0% by weight of the total composition are used.

In some embodiments, microcrystalline cellulose (MCC) can be used (e.g., carboxymethyl cellulose with sodium carboxymethyl cellulose). An example of a source of MCC is Avicel® (FMC Corporation), which contains MCC in combination with sodium carboxymethyl cellulose (NaCMC). Both Avicel®. RC-591 (MCC containing 8.3 to 13.8 weight % NaCMC) and Avicel®. CL-611 (MCC containing 11.3 to 18.8 weight % NaCMC) may be used in certain aspects. In certain embodiments, the ratio of microcrystalline cellulose to cellulose ether thickening agent is from 1:1 to 1:3 by weight; or from 1:1.5 to 1:2.75 by weight. In any of the above embodiments comprising sodium carboxymethylcellulose, microcrystalline cellulose may be used in combination with NaCMC. In certain such embodiments, the MCC/sodium carboxymethylcellulose may be present in an amount of from 0.5 to 1.5 weight % based on the total weight of the composition.

### Abrasives

Natural calcium carbonate is found in rocks such as chalk, limestone, marble and travertine. It is also the principle component of egg shells and the shells of mollusks. The natural calcium carbonate abrasive of the invention is typically a finely ground limestone which may optionally be refined or partially refined to remove impurities. For use in the present invention, the material has an average particle size of less than 10 microns, e.g., 3-7 microns, e.g. about 5.5 microns. For example a small particle silica may have an average particle size (D50) of 2.5-4.5 microns. Because natural calcium carbonate may contain a high proportion of relatively large particles of not carefully controlled, which may unacceptably increase the abrasivity, preferably no more than 0.01%, preferably no more than 0.004% by weight of particles would not pass through a 325 mesh. The material has strong crystal structure, and is thus much harder and more abrasive than precipitated calcium carbonate. The tap density for the natural calcium carbonate is for example between 1 and 1.5 g/cc, e.g., about 1.2 for example about 1.19 g/cc. There are different polymorphs of natural calcium carbonate, e.g., calcite, aragonite and vaterite, calcite being preferred for purposes of this invention. An example of a commercially available product suitable for use in the present invention includes Vicron® 25-11 FG from GMZ.

Precipitated calcium carbonate is generally made by calcining limestone, to make calcium oxide (lime), which can then be converted back to calcium carbonate by reaction with carbon dioxide in water. Precipitated calcium carbonate has a different crystal structure from natural calcium carbonate. It is generally more friable and more porous, thus having lower abrasivity and higher water absorption. For use in the present invention, the particles are small, e.g., having an average particle size of 1-5 microns, and e.g., no more than 0.1%, preferably no more than 0.05% by weight of particles which would not pass through a 325 mesh. The particles may for example have a D50 of 3-6 microns, for example 3.8=4.9, e.g., about 4.3; a D50 of 1-4 microns, e.g. 2.2-2.6 microns, e.g., about 2.4 microns, and a D10 of 1-2 microns, e.g., 1.2-1.4, e.g. about 1.3 microns. The particles have relatively high water absorption, e.g., at least 25 g/100 g, e.g. 30-70 g/100 g. Examples of commercially available products suitable for use in the present invention include, for example, Carbolag® 15 Plus from Lagos Industria Quimica.

In certain embodiments the invention may comprise additional calcium-containing abrasives, for example calcium phosphate abrasive, e.g., tricalcium phosphate (Ca₃(PO₄)₂), hydroxyapatite (Ca₁₀(PO₄)₆(OH)₂), or dicalcium phosphate dihydrate (CaHPO₄·2H₂O, also sometimes referred to herein as DiCal) or calcium pyrophosphate, and/or silica abrasives, sodium metaphosphate, potassium metaphosphate, aluminum silicate, calcined alumina, bentonite or other siliceous materials, or combinations thereof. Any silica suitable for oral care compositions may be used, such as precipitated silicas or silica gels. For example synthetic amorphous silica. Silica may also be available as a thickening agent, e.g., particle silica. For example, the silica can also be small particle silica (e.g., Sorbosil AC43 from PQ Corporation, Warrington, United Kingdom). However the additional abrasives are preferably not present in a type or amount so as to increase the RDA of the dentifrice to levels which could damage sensitive teeth, e.g., greater than 130.

### Amino Acids

In one aspect, the compositions of the disclosure, e.g., any of Compositions 1.0 et seq, include a basic amino acid. The basic amino acids which can be used in the compositions and methods of the invention include not only naturally occurring basic amino acids, such as arginine, lysine, and histidine, but also any basic amino acids having a carboxyl group and an amino group in the molecule, which are water-soluble and provide an aqueous solution with a pH of 7 or greater.

For example, basic amino acids include, but are not limited to, arginine, lysine, serine, citrullene, ornithine, creatine, histidine, diaminobutanoic acid, diaminoproprionic acid, salts thereof or combinations thereof. In a particular embodiment, the basic amino acids are selected from arginine, citrullene, and ornithine.

In certain embodiments, the basic amino acid is arginine, for example, L-arginine, or a salt thereof.

In another aspect, the compositions of the disclosure (e.g., any of Compositions 1.0 et seq) can further include a neutral amino acid (e.g., either alone or in combination with a basic amino acid), which can include, but are not limited to, one or more neutral amino acids selected from the group consisting of alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, and combinations thereof.

The compositions of the disclosure (e.g., Composition 1.0 et seq) are intended for topical use in the mouth and so salts for use in the present invention should be safe for such use, in the amounts and concentrations provided. Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form a physiological acceptable anion, e.g., hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion.

### Water

Water is present in the oral compositions of the disclosure, e.g., any of Composition 1.0 et seq. Water, employed in the preparation of commercial oral compositions should be deionized and free of organic impurities. Water commonly makes up the balance of the compositions and includes 5% to 45%, e.g., 10% to 20%, e.g., 25-35%, by weight of the oral compositions. This amount of water includes the free water which is added plus that amount which is introduced with other materials such as with sorbitol or silica or any components of the invention. The Karl Fischer method is a one measure of calculating free water.

### Humectants

Within certain embodiments of the oral compositions, e.g., any of Composition 1.0 et seq., it is also desirable to incorporate a humectant to reduce evaporation and also contribute towards preservation by lowering water activity. Certain humectants can also impart desirable sweetness or flavor to the compositions. The humectant, on a pure humectant basis, generally includes 15% to 70% in one embodiment or 30% to 65% in another embodiment by weight of the composition.

Suitable humectants include edible polyhydric alcohols such as glycerin, sorbitol, xylitol, propylene glycol as well as other polyols and mixtures of these humectants. Mixtures of glycerin and sorbitol may be used in certain embodiments as the humectant component of the compositions herein.

Flavorings for use in the present invention may include extracts or oils from flavorful plants such as peppermint, spearmint, cinnamon, wintergreen, and combinations thereof, cooling agents such as menthol, methyl salicylate, as well as sweeteners, which may include polyols (which also function as humectants), saccharin, acesulfame, aspartame, neotame, stevia and sucralose.

### EXAMPLES

Unless otherwise noted, the pH of all solutions described in the Examples is about 7. Unless otherwise noted, all figures for stannous ion concentration refer to soluble stannous, not total stannous (total stannous being soluble and insoluble stannous combined).

### Example 1—Stabilization of Stannous Fluoride in Aqueous Solution by Potassium Nitrate and Tetrasodium Pyrophosphate

Simple solutions of 0.454% stannous fluoride in water combined with different stabilizing agents are compared using visual observation and assays for soluble stannous ion concentration. As a baseline, a solution of 0.454% stannous fluoride in water is compared to a solution of 0.454% stannous fluoride and 5.0% potassium nitrate. Both solutions have a pH of 7. The solutions are aged at room temperature for 30 days, and soluble stannous ion content is measured at 1 day, 5 days, 9 days, 15 days, and 26 days. Stannous ion (Sn(II)) concentration is determined by titration. 0.1N iodine solution is first added to a sample of the solution and stirred for at least one hour. The solution is observed to turn brown. 0.1N sodium thiosulfate solution is then added until the mixture turns and remains stably white. The amount of soluble stannous ion is then calculated as the difference between the molar amount of iodine added and the molar amount of sodium thiosulfate added, and this molar amount of soluble stannous ion is converted to a concentration figure. The concentration value so determined is then converted to a percentage of the theoretical amount of stannous (II) which should be present based on the formulation of the solution.

The results are shown in the table below, expressed as the percentage of soluble stannous compared to the theoretical amount:

| | Day 1 | Day 5 | Day 9 | Day 15 | Day 26 |
|---|---|---|---|---|---|
| SnF₂ | 96% | 93% | 89% | 80% | 63% |
| SnF₂ + KNO₃ | 100% | 100% | 94% | 83% | 68% |

The results show that at neutral pH, potassium nitrate by itself improves stannous ion stability initially, but by day 9, stannous ion concentration continues to fall comparable to the unstabilized stannous fluoride solution. It is also observed that both solutions are initially turbid, and continued aging results in the solutions becoming yellow and remaining turbid. For comparison, a solution of SnF₂ at its native pH (acidic) is clear and colorless and remains so through aging. Thus, this demonstrates that a solution of stannous ion at near or above neutral pH is unstable, but that potassium nitrate provides short-lived stabilization.

In a second set of experiments, the stability of 0.454% stannous fluoride is compared in solutions which each comprise 0.3% potassium nitrate and optionally a second chelating agent. The second agent is selected from 0.77% tetrasodium pyrophosphate (TSPP), 2.2% sodium citrate, 1.0% sodium gluconate, and 0.5% arginine, and the resulting three-component solutions have a pH of 7 in each case. Each solution is clear, colorless and homogenous, except for the solution with arginine, which is initially turbid. 0.454% stannous fluoride in water is included as a negative control. As a positive control, one solution consists of 0.454% stannous fluoride and 0.3% potassium nitrate acidified to pH 3. As noted previously, it has been reported that at a pH below 6, potassium nitrate alone stabilizes stannous fluoride in solution, and that result is confirmed here. In this experiment, aging is conducted at 60° C. with stannous ion concentration measured at 0 days, 6 or 7 days and at 14 days. The results are shown in the table below, expressed as the percentage of soluble stannous compared to the theoretical amount:

| | Day 0 | Day 6 | Day 7 | Day 14 |
|---|---|---|---|---|
| SnF₂ + KNO₃ + TSPP | 100% | 96% | | 87% |
| SnF₂ + KNO₃, pH 3 | 98% | | 92% | 85% |
| SnF₂ + KNO₃ + citrate | 99% | 81% | | 55% |
| SnF₂ + KNO₃, + gluconate | 100% | 47% | | 44% |
| SnF₂ + KNO₃ + arginine | 100% | 29% | | 17% |
| SnF₂ | 99% | | 32% | 7% |

Stannous fluoride/potassium nitrate/TSPP solution remains homogenous at day 14, showing no signs of insoluble tin precipitation. The data demonstrates that absent a stabilizing agent, less than 10% of the original stannous ion remains available in solution after 14 days at 60° C. Potassium nitrate effectively stabilizes stannous ion under these conditions at a pH of 3, but not at neutral pH, as seen by comparing these results with the preceding results. Unexpectedly, however, the combination of potassium nitrate and TSPP at neutral pH stabilizes stannous as effectively as potassium nitrate alone at acidic pH. The same effect is not obtained using alternative chelating agents, such as citrate, gluconate and arginine. Thus, the particular combination of potassium nitrate and TSPP is shown to provide a synergistic or unexpected stabilizing effect on stannous ion.

While potassium nitrate is found to stabilize stannous ion at acidic pH, it is also found that the solution undergoes an undesirable discoloration at the same time. This is most apparent after 4 weeks of aging at 60° C. While the stannous fluoride/potassium nitrate/TSPP solution remains homogenous and colorless after 4 weeks, the stannous fluoride/potassium nitrate/pH 3 solution becomes clearly yellow. This is confirmed by comparing UV/Vis spectroscopy, which shows a peak at about 300-310 nm wavelength in the acidic solution, which is not present in the neutral solution with TSPP.

In a third experiment, the effect of sodium tripolyphosphate (STPP) is compared to the effect of TSPP in stabilizing stannous over 2 weeks of aging at 60° C. STPP provides comparable benefits to TSPP, and these are both demonstrated as being synergistic or unexpected effects resulting from the interaction of the potassium nitrate and the polyphosphate salt. The results are shown in the table below:

| Components (wt %) | | | | % Stannous, |
|---|---|---|---|---|
| SnF₂ | KNO₃ | TSPP | STPP | Day 14 |
| 0.454 | 0 | 0 | 0 | 7.5% |
| 0.454 | 0.3 | 0 | 0 | 9.0% |
| 0.454 | 0 | 0.77 | 0 | 37% |
| 0.454 | 0 | 0 | 1.07 | 32% |
| 0.454 | 0.3 | 0.77 | 0 | 87% |
| 0.454 | 0.3 | 0 | 1.07 | 93% |

### Example 2: Stability of Stannous Fluoride/Potassium Nitrate/TSPP Mixture Over a Range of Ratios

A series of comparative solutions comprising stannous fluoride, potassium nitrate and TSPP are prepared and subjected to aging for 14 days at 60° C. On day 14, soluble stannous ion concentration is measured and visual observations are made. All solutions have 0.454% stannous fluoride, and the amounts of potassium nitrate and TSPP are adjusted to arrive at the desired molar ratios. The results are shown in the table below:

| Molar Ratio | | | % Stannous, |
|---|---|---|---|
| SnF₂ | KNO₃ | TSPP | Day 14 |
| 1 | 1 | 0 | 9% |
| 1 | 1 | 0.07 | 29% (turbid) |
| 1 | 1 | 0.1 | 25% (turbid) |
| 1 | 1 | 0.3 | 83% (turbid) |
| 1 | 1 | 0.7 | 83% (turbid) |
| 1 | 1 | 1 | 87% |
| 1 | 1 | 1.3 | 87% |
| 1 | 1 | 2 | 78% |
| 1 | 1 | 2.6 | 73% |
| 1 | 1 | 5.2 | 59% |
| 1 | 0 | 1 | 37% |
| 1 | 0.04 | 1 | 52% |
| 1 | 0.1 | 1 | 73% |
| 1 | 0.2 | 1 | 75% |
| 1 | 0.3 | 1 | 80% |
| 1 | 0.7 | 1 | 86% |
| 1 | 1 | 1 | 87% |
| 1 | 2 | 1 | 86% |
| 1 | 3.4 | 1 | 88% |
| 1 | 7 | 1 | 85% |
| 1 | 12 | 1 | 83% |
| 1 | 17 | 1 | 84% |
| 1 | 34 | 1 | 76% |

A molar ratio of 1:1 stannous fluoride to potassium nitrate, a high level of stannous ion stability (>80%) and solution homogeneity can be obtained over a stannous fluoride to TSPP molar ratio of 1:1 to 1:2.5. When less TSPP is used, a precipitate forms even while maintaining acceptable stannous ion stability, while when the lowest or highest amounts of TSPP are employed, stannous ion stability drops.

It is further found that at a molar ratio of 1:1 stannous fluoride to TSPP, a high level of stannous ion stability (>80%) and solution homogeneity can be obtained over a wide range of stannous fluoride/potassium nitrate molar ratios.

Together these results further support the unique unexpected synergy between potassium nitrate and TSPP ins stabilizing stannous ion in aqueous solution.

### Example 3—Stability of Stannous Fluoride/Potassium Nitrate/STPP Mixture Over a Range of Ratios

To evaluate whether the same stabilization effect can be obtained using a tripolyphosphate salt, the same experimental procedure as outlined in Example 2 was repeated using sodium tripolyphosphate instead of tetrasodium pyrophosphate. The results are shown in the table below.

| Molar Ratio | | | % Stannous, |
|---|---|---|---|
| SnF₂ | KNO₃ | TSPP | Day 14 |
| 1 | 1 | 0 | 9% |
| 1 | 1 | 0.05 | 69% (turbid) |
| 1 | 1 | 0.1 | 84% (turbid) |
| 1 | 1 | 0.3 | 86% (turbid) |
| 1 | 1 | 0.5 | 90% (turbid) |
| 1 | 1 | 0.7 | 91% (turbid) |
| 1 | 1 | 1 | 92% |
| 1 | 1 | 1.5 | 92% |
| 1 | 1 | 2 | 87% |
| 1 | 1 | 3 | 87% |
| 1 | 1 | 4 | 83% |
| 1 | 1 | 6 | 82% |
| 1 | 1 | 8 | 79% |
| 1 | 1 | 10 | 79% |
| 1 | 0 | 1 | 32% |
| 1 | 0.03 | 1 | 74% |
| 1 | 0.1 | 1 | 84% |
| 1 | 0.2 | 1 | 86% |
| 1 | 0.3 | 1 | 91% |
| 1 | 1 | 1 | 93% |
| 1 | 2 | 1 | 95% |
| 1 | 3.4 | 1 | 96% |
| 1 | 7 | 1 | 95% |
| 1 | 17 | 1.5 | 92% |
| 1 | 34 | 1.5 | 86% |

As found with TSPP, the combination of STPP and potassium nitrate is found to result in stabilization of stannous over wide concentration ranges and ratios. It is further found that high stannous stability can be achieved using lower concentrations of STPP than for TSPP.

### Example 4—Additional Studies on Stannous/Nitrate/Phosphate Systems

Additional studies are performed using the same 14-day, 60° C. accelerated aging study design, in which variations are made in the concentrations and/or components of the tested solutions.

In one experiment, the stabilizing effect of potassium nitrate and TSPP or STPP on stannous chloride is compared to the effect on stannous fluoride. As shown in the table below, it is found that STPP is somewhat more effective in stabilizing stannous chloride than TSPP is, although both polyphosphates provide effective stabilization of both stannous salts.

| Components (wt %) | | | | | % Stannous, |
|---|---|---|---|---|---|
| SnF₂ | SnCl₂ | KNO₃ | TSPP | STPP | Day 14 |
| 0.454 | 0 | 0.3 | 0.77 | 0 | 87% |
| 0 | 0.64 | 0.3 | 0.77 | 0 | 79% |
| 0.454 | 0 | 0.3 | 0 | 1.07 | 93% |
| 0 | 0.64 | 0.3 | 0 | 1.07 | 93% |

In another experiment, sodium nitrate or potassium c on e are compared to potassium nitrate in order to further evaluate the role of potassium nitrate in stabilizing stannous. The results are shown in the table below. It is found that sodium nitrate provides a comparable stabilizing affect as potassium nitrate, whereas potassium chloride does not provide an additive stabilizing effect. The stannous stability obtained in an SnF₂/KCl/TSPP or SnF₂/KCl/STPP system is comparable to the results obtained above for an SnF₂/TSPP or SnF₂/STPP system, as shown in Example 1 (32% stannous at day 14 using STPP, and 37% using TSPP). Thus, it is apparent that the nitrate anion provides a unique stabilizing effect which is not obtained using the isoelectronic and comparably sized chloride anion. Moreover, it is seen that the choice of cation to the nitrate anion makes a negligible difference to the outcome.

| Components (wt %) | | | | | | % Stannous, |
|---|---|---|---|---|---|---|
| SnF₂ | KNO₃ | NaNO₃ | KCl | TSPP | STPP | Day 14 |
| 0.454 | 0.3 | 0 | 0 | 0.77 | 0 | 87% |
| 0.454 | 0 | 0.25 | 0 | 0.77 | 0 | 86% |
| 0.454 | 0 | 0 | 0.23 | 0.77 | 0 | 34% |
| 0.454 | 0.6 | 0 | 0 | 0 | 1.6 | 96% |
| 0.454 | 0 | 0.5 | 0 | 0 | 1.6 | 94% |
| 0.454 | 0 | 0 | 0.46 | 0 | 1.6 | 40% |

In another experiment, the initial concentration of stannous fluoride is varied to determine the range over which the KNO₃/polyphosphate system provides a stabilizing effect. Two stabilizing systems are evaluated: SnF₂/KNO₃/TSPP at a 1:1:1 molar ratio, and SnF₂/KNO₂/STPP at a 1:2:1 molar ratio. The results are shown in the table below. It is unexpectedly found that the KNO₃/TSPP system provides highly effective stabilizing over an initial stannous fluoride concentration range of 0.1 to 1.7%, but this efficiency drops at lower initial stannous fluoride concentrations. In contrast, the KNO₃/STPP system provides effective stabilization over the entire stannous fluoride concentration range tested.

| Components (wt %) | | | | % Stannous, |
|---|---|---|---|---|
| SnF₂ | KNO₃ | TSPP | STPP | Day 14 |
| 0.05 | 0.03 | 0.077 | 0 | 2% |
| 0.09 | 0.06 | 0.16 | 0 | 52% |
| 0.15 | 0.1 | 0.27 | 0 | 73% |
| 0.20 | 0.13 | 0.36 | 0 | 80% |
| 0.45 | 0.3 | 0.77 | 0 | 87% |
| 1.0 | 0.66 | 1.7 | 0 | 91% |
| 2.0 | 1.3 | 4.4 | 0 | 90% |
| 2.5 | 1.7 | 5.6 | 0 | 91% |
| 0.05 | 0.06 | 0 | 0.11 | 77% |
| 0.07 | 0.09 | 0 | 0.18 | 86% |
| 0.09 | 0.12 | 0 | 0.22 | 91% |
| 0.15 | 0.19 | 0 | 0.37 | 92% |
| 0.45 | 0.6 | 0 | 1.1 | 95% |
| 1.0 | 1.3 | 0 | 2.4 | 91% |
| 1.7 | 2.2 | 0 | 6.0 | 86% |

In an additional experiment, the stannous chloride/potassium nitrate/TSPP (1:1 stannous to nitrate, 1:1 or 1:1.5 stannous to TSPP) and the stannous chloride/potassium nitrate/STPP (1:2 stannous to nitrate, 1:1, 1:1.5 or 1:3 stannous to STPP) systems are evaluated at a different pH values. In order to achieve an initially clear, homogenous solution, a higher concentration of the polyphosphate is required at higher pH values (pH 8 or 9). At pH 9, the STPP-based system (1:2:3 molar ratio) is initially slightly turbid, but it becomes clear prior to the end of the study. It is unexpectedly found that the STPP-based system provides improved stabilization over the somewhat broader pH range compared to the TSPP-based system. The results are shown in the table below:

| Components (wt %) | | | | | % Stannous, |
|---|---|---|---|---|---|
| SnF₂ | KNO₃ | TSPP | STPP | pH | Day 14 |
| 0.454 | 0.3 | 0.77 | 0 | 6 | 84% |
| 0.454 | 0.3 | 0.77 | 0 | 7 | 87% |
| 0.454 | 0.3 | 1.2 | 0 | 8 | 68% |
| 0.454 | 0.6 | 0 | 1.07 | 6 | 95% |
| 0.454 | 0.6 | 0 | 1.07 | 7 | 95% |
| 0.454 | 0.6 | 0 | 1.6 | 8 | 94% |
| 0.454 | 0.6 | 0 | 3.2 | 9 | 76% |

### Example 5—Mouthwash Formulations

Exemplary representative mouthwash compositions according to the present disclosure are expected to be formulated as follows (quantities shown in % by weight of the composition):

| | Example number | | | |
|---|---|---|---|---|
| | A | B | C | D |
| Stannous Ion | 680 | 3400 | 340 | 340 |
| Concentration | | | | |
| (ppm) | | | | |
| SnF₂: KNO₃: TSPP | 1:1:1 | 1:1:1 | 1:1:1 | 1:4:1 |
| molar ratio | | | | |
| Water | Q.S. (~77) | Q.S. (~76) | Q.S. (~77) | Q.S. (~77) |
| L-Arginine | 1.5 | 1.5 | 1.5 | 1.5 |
| Nonionic surfactant (e.g., | 0.4 | 0.4 | 0.4 | 0.4 |
| Poloxamer 407) | | | | |
| Potassium Nitrate | 0.059 | 0.3 | 0.03 | 0.12 |
| Tetrasodium | 0.155 | 0.77 | 0.077 | 0.077 |
| Pyrophosphate | | | | |
| Glycerin | 7.5 | 7.5 | 7.5 | 7.5 |
| Sorbitol | 5.5 | 5.5 | 5.5 | 5.5 |
| Propylene Glycol | 7 | 7 | 7 | 7 |
| Flavors, Colors, and other | 0.168 | 0.168 | 0.168 | 0.168 |
| Minors | | | | |
| NaOH (50% Aqueous | 0.04 | 0.06 | 0.06 | 0.06 |
| Solution)) | | | | |

### Example 6—Dentifrice Formulations

Exemplary representative dentifrice compositions according to the present disclosure are expected to be formulated as follows (quantities shown in % by weight of the composition):

| | Example number | | | | | |
|---|---|---|---|---|---|---|
| | G | H | I | J | K | L |
| | Paste | Gel | Paste | Paste | Gel | Gel |
| Stannous Ion | 3400 | 3400 | 3400 | 3400 | 3400 | 3400 |
| Concentration (ppm) | | | | | | |
| SnF₂:KNO₃:TSPP/STPP | 1:1:1 | 1:1:1 | 1:17:1.6 | 1:17:1.9 | 1:10:1.4 | 1:10:1.7 |
| molar ratio | | | | | | |
| Water | Q.S. | Q.S. | Q.S. | Q.S. | Q.S. | Q.S. |
| | (e.g.,~20) | (e.g.,~12) | (e.g.,~20) | (e.g.,~20) | (e.g.,~12) | (e.g.,~12) |
| L-Arginine | 1.3 | 1.3 | 1.3 | 1.3 | 1.3 | 1.3 |
| Microcrystalline cellulose | 1 | 0 | 1 | 1 | 0 | 0 |
| (e.g., Avicel) | | | | | | |
| Polyethylene glycol (e.g., | 2 | 3 | 2 | 2 | 3 | 3 |
| PEG 600) | | | | | | |
| Xanthan Gum | 0.3 | 0 | 0.3 | 0.3 | 0 | 0 |
| Carboxymethyl cellulose | 0.8 | 0.65 | 0.8 | 0.8 | 0.8 | 0.8 |
| (e.g., Na CMC) | | | | | | |
| Stannous Fluoride | 0.454 | 0.454 | 0.454 | 0.454 | 0.454 | 0.454 |
| Potassium Nitrate | 0.3 | 0.3 | 5 | 5 | 3 | 3 |
| Tetrasodium | 0.77 | 0.77 | 1.2 | | 1.1 | |
| Pyrophosphate | | | | | | |
| Sodium Tripolyphosphate | | | | 2 | | 1.8 |
| Glycerin | 4 | 0 | 4 | 4 | 0 | 0 |
| Sorbitol | 44 | 55 | 39 | 38 | 52 | 52 |
| Silicas | 21.5 | 22.75 | 21.5 | 21.5 | 22.75 | 22.75 |
| Anionic surfactant (e.g., | 1.5 | 1.5 | 1.5 | 1.5 | 1.5 | 1.5 |
| SLS) | | | | | | |
| Zwitterionic surfactant | 1.25 | 1.25 | 1.25 | 1.25 | 1.25 | 1.25 |
| (e.g., betaine) | | | | | | |
| Flavors, Colors, and other | 1.8 | 1.75 | 1.8 | 1.8 | 1.75 | 1.75 |
| Minors | | | | | | |
| NaOH (50% Aqueous | 0.06 | 0.06 | 0 | 0 | 0 | 0 |
| Solution)) | | | | | | |
| Hydrochloric acid | 0 | 0 | 0.05 | 0.1 | 0 | 0.05 |

A further representative formula may be formulated as follows:

| | Example Formula M |
|---|---|
| | Paste |
| Stannous Ion Concentration (ppm) | 3400 |
| SnF₂: KNO₃: TSPP molar ratio | 1:10:1.6 |
| Water | Q.S. (~7) |
| L-Arginine | 1.5 |
| Polyethylene glycol (e.g., PEG 600) | 2 |
| Xanthan Gum | 0.4 |
| Stannous Fluoride | 0.454 |
| Potassium Nitrate | 3 |
| Tetrasodium Pyrophosphate | 1.2 |
| Sorbitol | 57 |
| Silicas | 23 |
| Anionic surfactant (e.g., SLS) | 1.5 |
| Zwitterionic surfactant (e.g., betaine) | 1.25 |
| Flavors, Colors, and other Minors | 2 |

### Example 7—Transparent Dentifrice Formulations

It is also expected that compositions made according to the present disclosure, especially toothpaste or gel compositions, are surprisingly translucent. Without being bound by theory, it is believed that the presence of un-solubilized stannous ion in a high-water dentifrice may contribute significantly to opacity. It therefore believed that the solubilization of stannous ion according to the present disclosure (by interaction with nitrate and polyphosphate ions) removes this impediment to clarity and transparency. It is expected that a properly formulated dentifrice composition according to the present disclosure will achieve substantial improvements in clarity and transparency compared to prior art dentifrice compositions.

### Example 8—ATP Production in Biofilm and Bacterial Community Shift

Formulas of the present invention are observed in an in vitro biofilm model that mimics 5 days' toothpaste usage twice daily. Saliva-derived biofilms are grown on hydroxyapatite discs held in a vertical position using a specially designed steel lid. An example of the laboratory technique described by Exterkate RAM, Crielaard W, Ten Cate JM. Different Response to Amine Fluoride by Streptococcus mutans and Polymicrobial Biofilms in a Novel High-Throughput Active Attachment Model. Caries Research. 2010. pp. 372-379, the contents of which are incorporated herein by reference. Sterilized discs are inoculated with 1.5 ml of 25% saliva in SHI medium and allowed to incubate for 4 h to allow for initial adhesion of bacteria. After 4 h, samples are treated for 2 min with 1:1 slurries of dentifrice:water and vigorously washed. Treated samples are transferred to fresh SHI medium and incubated overnight at 37° C., 5% CO₂. Samples are treated twice per day, with a minimum of four hours between treatments for the next 3 days. On the fifth day, samples are treated one time and then allowed to recover for at least 4 h in the incubator. Biofilms were harvested from discs by sonication and pellets were frozen and stored for further analysis via sequencing of the V3-V4 region of the 16s ribosomal subunit.

At the macroscopic level, treatments are studied for their impact on the overall metabolic activity of biofilms by measuring ATP production from biofilms using the BacTiter Glo luminescent assay (Promega). ATP production is reported as a percent reduction relative to biofilms treated with a placebo dentifrice.

TABLE 1

| | Percent Reduction |
|---|---|
| | of Bacterial ATP vs. |
| | Placebo (Negative |
| Test Sample | Control) |
| Test Sample 1 | 61.8% |
| (Stannous fluoride) | |
| Test Sample 2 | 27.9% |
| (Potassium Nitrate) | |
| Test Sample 3 | 62.6% |
| (Stannous fluoride and Potassium nitrate) | |
| Test Sample 4 | 84.9% |
| (Stannous fluoride and arginine) | |
| Test Sample 5 | 85.6% |
| Stannous fluoride, arginine, and | |
| potassium nitrate) | |

Components of the Test Samples listed in Table 1 above:

| | Test | Test | Test | Test | Test |
|---|---|---|---|---|---|
| | Sample | Sample | Sample | Sample | Sample |
| | 1 | 2 | 3 | 4 | 5 |
| Water | Q.S. | Q.S. | Q.S. | Q.S. | Q.S. |
| | (~7) | (~7) | (~7) | (~7) | (~7) |
| L-Arginine | — | — | — | 1.5 | 1.5 |
| Polyethylene glycol | 2 | 2 | 2 | 2 | 2 |
| (e.g., PEG 600) | | | | | |
| Xanthan Gum | 0.4 | 0.4 | 0.4 | 0.4 | 0.4 |
| Stannous Fluoride | 0.454 | — | 0.454 | 0.454 | 0.454 |
| Potassium Nitrate | — | 3 | 3 | — | 3 |
| Tetrasodium | 1.2 | 1.2 | 1.2 | 1.2 | 1.2 |
| Pyrophosphate | | | | | |
| Sorbitol | 59 | 62 | 59 | 60 | 57 |
| Silicas | 23 | 23 | 23 | 23 | 23 |
| Anionic surfactant | 1.5 | 1.5 | 1.5 | 1.5 | 1.5 |
| (e.g., SLS) | | | | | |
| Zwitterionic | 1.25 | 1.25 | 1.25 | 1.25 | 1.25 |
| surfactant | | | | | |
| (e.g., betaine) | | | | | |
| Flavors, Colors, | 2 | 2 | 2 | 2 | 2 |
| and other Minors | | | | | |

Using the biofilms described above, the ability of the test formulas to shift the composition of the bacterial communities in each treatment is studied. Oral biofilms are complex communities with up to 700 different species that can be present. The most efficient means of studying community shifts is to sequence the variable region of 16s rRNA subunit and use these sequences to make taxonomic assignments and identify the relative abundance of each genus/species within a community. A relatively large shift in the bacterial community of the biofilms is observed in the untreated biofilm samples when compared to biofilm samples treated with Test Sample 5 (e.g. stannous fluoride, arginine, and potassium nitrate). Relative to the untreated samples, the increased presence of Neisseria bacterial species can be observed in biofilm samples treated with arginine and/or potassium nitrate. Neisseria bacterial species is of particular interest given that it is known to be involved in nitrate reduction.

Biofilms are harvested from the HAP discs and total DNA is extracted. Relative abundances are obtained by sequencing the V3-V4 hypervariable region of the 16s rRNA gene, and the results are described in Table 2 below. These sequences are used to identify bacteria present in hl community by comparison to the HOMD database of oral microbes. With the exception of Veillonella and Prevotella species, which appear to make up the majority of bacterial genera represented in the untreated biofilm samples, a number of genera associated with nitrate reduction appear to have an increased presence in the treated biofilm samples indicating a shift in the bacterial community representation:

TABLE 2
Relative Abundance of Bacterial Genera in Biofilms harvested from HAP discs

| Sample | A* | B | C | D | E | F | G | H | I | J | K | L |
|---|---|---|---|---|---|---|---|---|---|---|---|---|
| Untreated | — | — | — | 31.1 | — | 0.2 | — | — | — | 38.6 | — | 0.1 |
| (Placebo - | | | | | | | | | | | | |
| Negative | | | | | | | | | | | | |
| Control) | | | | | | | | | | | | |
| Test Sample 1 | 1.8 | 1.4 | — | 40.4 | — | 0.2 | — | — | — | 0.6 | 8.5 | 0.1 |
| (Stannous | | | | | | | | | | | | |
| fluoride) | | | | | | | | | | | | |
| Test Sample 2 | 0.1 | 4.8 | — | 28.5 | 0.1 | 0.3 | — | — | — | 27.7 | 0.1 | 0.1 |
| (Potassium | | | | | | | | | | | | |
| Nitrate) | | | | | | | | | | | | |
| Test Sample 3 | 2.2 | 7.8 | — | 35.2 | — | 0.1 | — | — | — | 6.3 | 3.9 | 0.1 |
| (Stannous | | | | | | | | | | | | |
| fluoride and | | | | | | | | | | | | |
| Potassium | | | | | | | | | | | | |
| nitrate) | | | | | | | | | | | | |
| Test Sample 4 | 0.7 | 0.9 | — | 33.0 | — | 0.3 | — | — | — | 0.2 | 0.8 | 0.8 |
| (Stannous | | | | | | | | | | | | |
| fluoride and | | | | | | | | | | | | |
| arginine) | | | | | | | | | | | | |
| Test Sample 5 | 2.2 | 4.6 | — | 26.5 | — | 0.1 | 0.9 | — | — | 0.1 | 1.4 | 0.5 |
| (Stannous | | | | | | | | | | | | |
| fluoride, | | | | | | | | | | | | |
| arginine, and | | | | | | | | | | | | |
| potassium | | | | | | | | | | | | |
| nitrate) | | | | | | | | | | | | |

(*“a” = Haemophilus; “b” = Neisseria; “c” = Eikonella; “d” = Veillonella; “e” = Selemononas; “f” = Granulicatella; “g” = Staphyloccocus; “h” = Bacillus; “i” = Capnocytophaga; “j” = Prevotella; “k” = Rothia; “l” = Actinomyces)

1. An oral care composition comprising
(i) stannous fluoride or stannous chloride or stannous pyrophosphate;
(ii) nitric acid, a water-soluble nitrate salt, or a combination thereof;
(iii) a water-soluble alkali metal polyphosphate;
(iv) a basic amino acid; and
(v) about 10 wt. % or more of water, by weight of the oral care composition.

2. The oral care composition of claim 1, wherein the water-soluble nitrate salt is selected from an alkali or alkaline earth metal nitrate, zinc nitrate, silver nitrate, ammonium nitrate, and a combination of two or more thereof.

3. The oral care composition of claim 1, wherein the water-soluble nitrate salt is selected from lithium nitrate, sodium nitrate, potassium nitrate, magnesium nitrate, calcium nitrate, and a combination of two or more thereof.

4. The oral care composition of claim 1, wherein the water-soluble nitrate salt is potassium nitrate.

5. The oral care composition of claim 1, wherein the water-soluble alkali metal polyphosphate is selected from a pyrophosphate, tripolyphosphate, tetraphosphate, hexametaphosphate, and a combination of two or more thereof.

6. The oral care composition of claim 5, wherein the water-soluble alkali metal polyphosphate is selected from sodium pyrophosphate, potassium pyrophosphate, sodium tripolyphosphate, potassium tripolyphosphate, and a combination of two or more thereof.

7. The oral care composition of claim 1, wherein the water-soluble nitrate salt is potassium nitrate and the water-soluble alkali metal polyphosphate salt is tetrasodium pyrophosphate.

8. The oral care composition of claim 1, wherein the oral care composition comprises a molar ratio of alkali metal polyphosphate to the stannous ion source of about 1:1 to about 5:1.

9. The oral care composition of claim 1, wherein the oral care composition comprises a molar ratio of the total amount of nitric acid and/or water-soluble nitrate salt to the total amount of stannous ion source of about 0.3:1 to about 20:1.

10. The oral care composition of claim 1, wherein the composition comprises from about 0.1 to about 2 wt. % of the stannous ion source, by weight of the composition; from about 0.1 to about 5 wt. % of the nitric acid, the water-soluble nitrate salt, or the combination of two or more thereof, by weight of the composition; from about 0.1 to about 5 wt. % of the alkali metal polyphosphate salt, by weight of the oral care composition.

11. The oral care composition of claim 1, wherein the composition comprises from about 10 to about 95 wt. % of water, by weight of the oral care composition.

12. (canceled)

13. (canceled)

14. The oral care composition of claim 1, wherein the composition comprises one or more humectants in an amount of from about 5 to about 25 wt. %, by weight of the oral care composition.

15. The oral care composition claim 1, wherein the basic amino acid is selected from the following: arginine, glycine, lysine, serine, citrullene, ornithine, creatine, histidine, diaminobutanoic acid, diaminoproprionic acid, and combinations thereof.

16. The oral care composition of claim 15, wherein the basic amino acid comprises arginine, glycine, lysine, or a combination of two or more thereof.

17. The oral care composition of 16, wherein the amount of arginine is from about 1 to about 15 wt. %, by weight of the oral care composition.

18. The oral care composition of claim 1 further comprising about 0.01 to about 0.09 wt. % of charcoal, by weight of the oral care composition; wherein the composition is formulated as a dentifrice.

19. The oral care composition of claim 1, wherein the oral care composition is in the form of a single component, single phase oral care composition.

20. (canceled)

21. (canceled)

22. A method of treatment or prevention of gingivitis, plaque, dental caries, and/or dental hypersensitivity, the method comprising the application to the oral cavity of a person in need thereof, of a composition according to claim 1.

23. A method of stabilizing stannous ion in an aqueous oral care composition comprising the steps of (1) providing an aqueous vehicle, (2) adding to the aqueous vehicle a stannous ion source, (3) adding to the aqueous vehicle a nitrate ion source, and (4) adding to the aqueous vehicle a polyphosphate ion source, wherein the final composition is a single-component high-water composition.

24. A method of treating or reducing systemic blood pressure, in a subject in need thereof, wherein the method comprises administering an oral care composition of claim 1 to the oral cavity of a person in need thereof.

25. (canceled)

26. (canceled)
